SEQUENCE LISTING (36 sequences)

SEQ ID NO: 1 - skipped

SEQ ID NO: 2 - protein, 17 aa, synthetic construct
LINPYNGXTTYNXKFXG

SEQ ID NO: 3 - protein, 8 aa, Mus musculus
EGNWEGVY

SEQ ID NO: 4 - protein, 16 aa, synthetic construct
XSSQSLLDSNGKTYLH

SEQ ID NO: 5 - protein, 7 aa, synthetic construct
LVSXLDS

SEQ ID NO: 6 - protein, 9 aa, Mus musculus
VQGTHFPFT

SEQ ID NO: 7 - protein, 5 aa, Mus musculus
GFTMN

SEQ ID NO: 8 - protein, 17 aa, Mus musculus
LINPYNGVTTYNQKFKG

SEQ ID NO: 9 - protein, 7 aa, Mus musculus
LVSILDS

SEQ ID NO: 10 - protein, 16 aa, synthetic construct
RSSQSLLDSNGKTYLH

SEQ ID NO: 11 - protein, 16 aa, Mus musculus
KSSQSLLDSNGKTYLH

SEQ ID NO: 12 - protein, 5 aa, Mus musculus
GYTLN

SEQ ID NO: 13 - protein, 17 aa, Mus musculus
LINPYNGITTYNPKFMG

SEQ ID NO: 14 - protein, 7 aa, Mus musculus
LVSKLDS

SEQ ID NO: 15 - protein, 117 aa, synthetic construct
EVQLVQSGAEVVKPGASVRLSCKASGYSFTGFTMNWVRQALGQGLEWMGLINPYNGVTTYNQKFKGRLTMTADMSTRTVYMDLSSLRYEDTAVYYCTREGNWEGVYWGQGTLVTVSS

SEQ ID NO: 16 - protein, 117 aa, synthetic construct
EVQLVQSGAEVKKPGASVRLSCKASGYSFTGFTMNWVRQALGQGLEWMGLINPYNGVTTYNQKFKGRLTMTADMSTRTVYMDLSSLRYEDTAVYYCTREGNWEGVYWGQGTLVTVSS

SEQ ID NO: 17 - protein, 117 aa, synthetic construct
EVQLVQSGAEVKKPGASVRLSCKASGYSFTGFTMNWVRQALGQGLEWMGLINPYNGVTTYNQKFKGRLTMTRDMSTRTVYMDLSSLTYEDTAVYYCTREGNWEGVYWGQGTLVTVSS

SEQ ID NO: 18 - protein, 117 aa, synthetic construct
EVQLVQSGAEVVKPGASVRLSCKASGYSFTGFTMNWVRQALGQGLEWMGLINPYNGVTTYNQKFKGRLTMTRDMSTRTVYMDLSSLTYEDTAVYYCTREGNWEGVYWGQGTLVTVSS

SEQ ID NO: 19 - protein, 117 aa, synthetic construct
EVQLVQSGAEVVKPGASVRLSCKASGYSFTGFTMNWVRQALGQGLEWMGLINPYNGVTTYNQKFKGRLTMTADMSTRTVYMDLSSLTYEDTAVYYCTREGNWEGVYWGQGTLVTVSS

SEQ ID NO: 20 - protein, 117 aa, synthetic construct
EVQLVQSGAEVKKPGASVRLSCKASGYSFTGFTMNWVRQALGQGLEWMGLINPYNGVTTYNQKFKGRLTMTADMSTRTVYMDLSSLTYEDTAVYYCTREGNWEGVYWGQGTLVTVSS

SEQ ID NO: 21 - protein, 117 aa, synthetic construct
QVQLVQSGPEVKKPGSSVKVSCKASGYSFTGFTMNWVRQTPGKGLEWIGLINPYNGVTTYNQKFKGRVTITADESTRTAYMELLSLTYEDTAVYYCTREGNWEGVYWGQGTPVTVSA

SEQ ID NO: 22 - protein, 117 aa, synthetic construct
EVQLVQSGAEVKKPGASVRLSCKASGYSFTGFTMNWVRQALGQGLEWMGLINPYNGVTTYNQKFKGRLTMTRDMSTRTVYMDLSSLRYEDTAVYYCTREGNWEGVYWGQGTLVTVSS

SEQ ID NO: 23 - protein, 112 aa, synthetic construct
EIVLTQSPLSLSVTLGQSASISCRSSQSLLDSNGKTYLHWFILRPGQSPRRLIYLVSILDSGVPDRFSGSGSGTDFTLKISRVEAEDVGVYYCVQGTHFPFTFGSGTKLEIK

SEQ ID NO: 24 - protein, 112 aa, synthetic construct
EVVLTQSPLSISVTLGQSASISCRSSQSLLDSNGKTYLHWFILRPGQSPRRLIYLVSILDSGIPDRFSGSGSGTDFTLKISRVEAEDVGVYYCVQGTHFPFTFGGGTKLEIK

SEQ ID NO: 25 - protein, 117 aa, Mus musculus
EVQLQQSGPELVKPGTSIKMSCKASGYSFTGFTMNWVKQSHGKNLEWIGLINPYNGVTTYNQKFKGKATITVDKSSRTAYMELLSLTYEDSAVYYCTREGNWEGVYWGQGTPVTVSA

SEQ ID NO: 26 - protein, 112 aa, Mus musculus
EVVLTQTPLTLSVTIGQSASISCKSSQSLLDSNGKTYLHWFLLRPGQSPKRLIYLVSILDSGVPDRFTGSGSGTDFTLKISRVEAEDLGIYYCVQGTHFPFTFGSGTKLEIK

SEQ ID NO: 27 - protein, 117 aa, Mus musculus
KVQLQQSGPELVKPGTSIKMSCKTSGYSFTGYTLNWVKQSPGKNPEWIGLINPYNGITTYNPKFMGKATLTVDKSSSTAYMELLSLTSEDSAVYYCSREGNWEGVYWGQGTLVTVSA

SEQ ID NO: 28 - protein, 112 aa, Mus musculus
DVVMTQTPLTLSVTIGQPASISCKSSQSLLDSNGKTYLHWLLLRPGQSPKRLIYLVSKLDSGVPDRFTGSGSGTDFTLKISRVEAEDLGVYFCVQGTHFPFTFGSGTKLEIK

SEQ ID NO: 29 - protein, 335 aa, Mus musculus
AKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIESRRPIPPNSCPPCKECSIFPAPDLLGGPSVFIFPPKIKDVLMISLSPIVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITDFLPAEIAVDWTSNGHKELNYKNTAPVLDTDGSYFMYSKLRVQKSTWEKGSLFACSVVHEGLHNHHTTKTISRSLGK

SEQ ID NO: 30 - protein, 107 aa, Mus musculus
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 31 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 107 aa, Homo sapiens [sequence omitted]

SEQ ID NO: 33 - protein, 444 aa, synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 219 aa, synthetic construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 451 aa, synthetic construct
EVQLLESGGGLVQPGGSLRLSCAASGFTFSSYPMSWVRQAPGKGLEWVSAISGSGGSTYYADSVKGRFTISRDNSKNTLYLQMNSLRAEDTAVYYCAREGGSGSYYNGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 214 aa, synthetic construct
DIQMTQSPSTLSASVGDRVTITCRASQSLGNWLAWYQQKPGKAPKLLIYQASTLESGVPSRFSGSGSGTEFTLTISSLQPDDFATYYCQHYKGSFWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0217804 A1
(43) Pub. Date: Jul. 30, 2026

(54) ANTIBODY WHICH BINDS TO ABETAPE3

(71) Applicant: BIOARCTIC AB, Stockholm (SE)

(72) Inventors: Paulina APPELKVIST, Stockholm (SE); Emma BOSTRÖM, Stockholm (SE); Christer MÖLLER, Stockholm (SE); Adeline RACHALSKI, Stockholm (SE); Jessica SIGVARDSON, Stockholm (SE); Linda SÖDERBERG, Stockholm (SE); Karin TEGERSTEDT, Stockholm (SE); James MCCLORY, Banbridge (GB)

(21) Appl. No.: 19/140,820

(22) PCT Filed: Dec. 22, 2023
(86) PCT No.: PCT/EP2023/087717

§ 371 Date: Jun. 18, 2025

(30) Foreign Application Priority Data
Dec. 22, 2022 (EP) 22215982.4

(51) Int. Cl. C07K 16/18; A61K 39/00; A61P 25/28

(52) U.S. Cl. CPC C07K 16/18; A61P 25/28; A61K 2039/505; C07K 2317/24; C07K 2317/565; C07K 2317/76; C07K 2317/92; C07K 2317/94

(57) ABSTRACT

Disclosed is an antibody or antigen-binding fragment thereof, which binds to AβpE3, i.e. to an N-terminally truncated and pyroglutamate-modified form of amyloid beta (Aβ), and therapeutic and diagnostic uses thereof.

### CROSS-REFERENCE TO RELATED APPLICATIONS

This application is a U.S. National Stage Application under 35 U.S.C. § 371 of International Patent Application No. PCT/EP2023/087717, filed Dec. 22, 2023, which claims benefit of priority of European Patent Application No. 22215982.4, filed Dec. 22, 2022, the disclosure of each of which is incorporated by reference herein in its entirety.

### SEQUENCE LISTING

This application contains an electronic Sequence Listing which has been submitted in XML file format via Patent Center, the entire content of which is incorporated by reference herein in its entirety. The Sequence Listing XML file submitted via Patent Center is entitled “14247-866-999_SL.xml”, was created on Jun. 10, 2025, and is 34,855 bytes in size.

### FIELD OF THE INVENTION

The present invention relates to an antibody or antigen-binding fragment thereof which binds to AβpE3, i.e. to an N-terminally truncated and pyroglutamate-modified form of amyloid beta (Aβ), and to therapeutic and diagnostic uses thereof.

### BACKGROUND

Alzheimer's disease (AD) is a progressive neurodegenerative dementia disorder which exists in a more common late-onset form and an early-onset familial form. AD is characterized by progressive loss of memory and cognitive function. At present, AD treatments are limited to symptomatic management and the prognosis is poor for AD patients. It is estimated that about 18 million people worldwide are presently suffering from AD, and the number of people suffering from AD is expected to increase due to the aging population. The prevalence of AD doubles approximately every 5 years from the age of 60, from 10% of individuals at the age of 65 to 50% of individuals at the age of 85 or more (Solomon (2007), Expert Opin Investig Drugs 16(6):819-828).

Accumulation of Aβ peptide in the brain is thought to play an important role in the neuropathology of AD. Aβ is generated from the amyloid precursor protein (APP) by sequential proteolysis and secreted via major regulated as well as minor constitutive secretory pathways. Aβ is a normal product of cell metabolism, which is present in the plasma and cerebrospinal fluid in healthy individuals. However, abnormal and excessive accumulation of Aβ in the brain leads to the formation of toxic Aβ aggregates that induce synaptic dysfunction and neuronal loss.

The main variants of Aβ detected in the human brain are Aβ1-40 and Aβ1-42. However, a significant proportion of AD brain Aβ also consists of N-terminally truncated species (Aβn−40/42 where n=2 to 11). Most such N-truncated Aβ peptides have been considered to be degradation products of full-length Aβ. It has been demonstrated that amyloid aggregates in AD brain and in brain of cognitively normal elderly subjects have different compositions, and that the toxic effect of these aggregates is correlated with the predominance of the N-terminal truncated species over the full-length Aβ.

Pyroglutamate-modified Aβ peptides have been demonstrated to be the predominant components among all N-terminally truncated Aβ species in AD brain. In particular AβpE3, an Aβ peptide having an amino-terminal pyroglutamate at position 3, has been shown to be a major N-truncated/modified constituent of intracellular, extracellular and vascular Aβ deposits in AD brain tissue. Furthermore, it has been demonstrated that AβpE3 progressively accumulates in the brain at the earliest stages of AD even before the appearance of clinical symptoms, which suggests that this peptide plays an important role in the formation of pathological amyloid aggregates. Thus, N-terminally truncated/modified Aβ peptides represent highly desirable and abundant therapeutic targets. This is particularly the case for AβpE3. For review and further references, see Perez-Garmendia and Gevorkian (2013), Curr Neuropharmacol 11:491-498.

Therapeutic antibodies against AβpE3 have been proposed, e.g. in WO2011/001366, WO2012/021469, WO2017/123517, WO2018/194951, WO2010/009987, WO2017/009459, WO2019/149689, WO2020/070225, WO2018/083628 and WO2020/193644.

Despite the existence of candidate antibodies within the field, no product has yet been granted regulatory approval, and there remains a need in the art for novel therapeutic, prophylactic, diagnostic and prognostic tools for detecting and treating AD and other neurodegenerative diseases.

### DESCRIPTION OF THE INVENTION

One object of the invention is to provide antibodies, or antigen-binding fragments thereof, having a novel and useful binding specificity.

Another object of the invention is to provide novel candidate antibodies for the treatment of neurodegenerative diseases via targeting of the AβpE3 peptide with a beneficial and unique binding profile.

Another object of the invention is to enable the diagnosis of AD and other neurodegenerative disorders via detection of AβpE3 implicated in disease formation and/or progression.

Another object of the invention is to provide antibodies that bind to the AβpE3 peptide with high affinity.

Another object of the invention is to provide antibodies that bind to the AβpE3 peptide with high specificity.

Another object of the invention is to provide antibodies that bind to the AβpE3 peptide with high selectivity with respect to other Aβ peptide variants.

Another object of the invention is to provide antibodies that bind to monomeric forms of AβpE3 as well as to putatively neurotoxic protofibril forms comprising AβpE3.

Another object of the invention is to provide antibodies that bind to both monomeric forms of AβpE3 and to the putatively neurotoxic protofibrils comprising AβpE3.

Another object of the invention is to provide antibodies that bind to all forms of AβpE3, including fibrils and plaques comprising AβpE3.

Another object of the invention is to provide AβpE3-binding antibodies that combine desirable properties for development into a biopharmaceutical product.

Another object of the invention is to provide AβpE3-binding antibodies that exhibit little or no immunogenicity upon administration in human subjects.

Another object of the invention is to provide AβpE3-binding antibodies that show a beneficial pharmacokinetic profile upon administration in human subjects, for example evidenced by one or more of a long half-life, a high total exposure and a low clearance.

One or more of these objects, and other objects that are apparent to the skilled person from reading the entire disclosure, are met by the various aspects disclosed.

Thus, in a first aspect, the present disclosure provides an antibody or antigen-binding fragment thereof, which has affinity for AβpE3, and in which the six complementarity determining regions (CDR) of the heavy chain variable domain (VH) and the light chain variable domain (VL) consist of the following amino acid sequences:

VH-CDR1:
(SEQ ID NO: 1)
GX₁TX₂N

wherein
X₁ is selected from Y and F; and
X₂ is selected from L and M;

VH-CDR2:
(SEQ ID NO: 2)
LINPYNGX₃TTYNX₄KFX₅G

wherein
X₃ is selected from I and V
X₄ is selected from P and Q; and
X₅ is selected from M and K;

VH-CDR3:
(SEQ ID NO: 3)
EGNWEGVY
VL-CDR1:
(SEQ ID NO: 4)
X₆SSQSLLDSNGKTYLH

wherein
X₆ is selected from K and R;

VL-CDR2:
(SEQ ID NO: 5)
LVSX₇LDS

wherein
X₇ is selected from I and K;

VL-CDR3:
(SEQ ID NO: 6)
VQGTHFPFT

In a second aspect, the present disclosure provides a pharmaceutical composition comprising an antibody or antigen-binding fragment thereof in accordance with the first aspect of the invention and a pharmaceutically acceptable excipient or carrier.

In further aspects, the present invention provides antibodies, antigen-binding fragments thereof and/or pharmaceutical compositions comprising the same for use in methods of treatment or for use in methods of detection or diagnosis as described herein.

### Anti-AβpE3 Antibodies

As described above, in a first aspect, the disclosure provides an antibody or antigen-binding fragment thereof, which has affinity for AβpE3, and in which the six CDRs of the VH and VL domain are as defined above with reference to SEQ ID NO:1-6.

The invention is based on detailed insights into the pathophysiology of diseases characterized by amyloid aggregation, and the identification of particular forms of Aβ in brain tissue from patients suffering from such diseases. As a non-limiting example, soluble forms of AβpE3 were found in extracts from AD brains, which further highlights the importance of obtaining antibodies that bind such species in a specific and/or selective manner. However, these insights also point to the potential benefits of having antibodies that bind AβpE3 in all forms that are present in connection with disease. These insights have enabled the generation of inventive antibodies, or antigen-binding fragments thereof, that are specific and/or selective for AβpE3 in its various forms. Also enabled was further development of the initial antibodies into humanized antibodies and variants thereof with a number of beneficial properties, including an unexpectedly favorable pharmacokinetic profile. Generation and characterization of exemplary such antibodies is detailed in Examples 1-14.

Without wishing to be bound by theory, it is contemplated that such novel antibodies, or antigen-binding fragments thereof, are useful in the diagnosis, prognosis and/or treatment of neurodegenerative diseases such as AD, through specific binding to the putatively disease-causing Aβ variant AβpE3.

As defined herein, embodiments of the antibody or antigen-binding fragment thereof of the first aspect of the disclosure are characterized by specific amino acid sequences in the regions determining its binding capability, such as the CDRs of the heavy and/or light chain variable domain, or indeed the entire VL and/or VH domains or regions. Non-limiting examples of such specific amino acid sequences are provided herein for the specific antibodies generated as described in Examples 1-14. It is contemplated that the specific sequence information provided for the generated antibodies enables the skilled person to define combinations and variations of these sequences within the scope of the invention.

Thus, in one embodiment of the first aspect, the antibody or antigen-binding fragment thereof comprises VH-CDR1, VH-CDR2 and VL-CDR2 regions which consist of the following amino acid sequences:

VH-CDR1:
(SEQ ID NO: 7)
GFTMN
VH-CDR2:
(SEQ ID NO: 8)
LINPYNGVTTYNQKFKG
VL-CDR2:
(SEQ ID NO: 9)
LVSILDS.

In a more specific embodiment, the antibody or antigen-binding fragment thereof comprises a heavy chain variable domain and a light chain variable domain, wherein said heavy chain variable domain comprises an amino acid sequence selected from the group consisting of SEQ ID NO:15-22 and amino acid sequences having at least 80% identity to any one of SEQ ID NO:15-22, provided that the three VH-CDR regions consist of SEQ ID NO:7, SEQ ID NO:8 and SEQ ID NO:3. For example, the amino acid sequence comprised in the VH in such an antibody or antigen-binding fragment thereof is selected from the group consisting of SEQ ID NO:15-21, such as selected from the group consisting of SEQ ID NO:15-16 and 18-21 or selected from the group consisting of SEQ ID NO:15-20, such as selected from the group consisting of SEQ ID NO:15-16 and 18-20, in particular selected from the group consisting of SEQ ID NO:15 and 18, most particularly being SEQ ID NO:18, or a sequence having at least 80% identity to any one of the listed subgroups, always provided that the three VH-CDR regions consist of SEQ ID NO:7, SEQ ID NO:8 and SEQ ID NO:3.

In another embodiment, the antibody or antigen-binding fragment thereof comprises a VL-CDR1 consisting of the amino acid sequence

(SEQ ID NO: 10)
RSSQSLLDSNGKTYLH.

In a more specific such embodiment, the antibody or antigen-binding fragment thereof comprises a heavy chain variable domain and a light chain variable domain, wherein said light chain variable domain comprises an amino acid sequence selected from the group consisting of SEQ ID NO:23-24 and amino acid sequences having at least 80% identity to any one of SEQ ID NO:23-24, provided that the three VL-CDR regions consist of SEQ ID NO:10, SEQ ID NO:9 and SEQ ID NO:6. For example, the amino acid sequence comprised in the VL in such an antibody or antigen-binding fragment thereof is SEQ ID NO:23 or a sequence having at least 80% identity to SEQ ID NO:23.

In one embodiment, the antibody or antigen-binding fragment thereof comprises both
a heavy chain variable domain comprising an amino acid sequence selected from the group consisting of SEQ ID NO:15-22 and amino acid sequences having at least 80% identity to any one of SEQ ID NO:15-22, provided that the three VH-CDR regions consist of SEQ ID NO:7, SEQ ID NO:8 and SEQ ID NO:3; and
a light chain variable domain comprising an amino acid sequence selected from the group consisting of SEQ ID NO:23-24 and amino acid sequences having at least 80% identity to any one of SEQ ID NO:23-24, provided that the three VL-CDR regions consist of SEQ ID NO:10, SEQ ID NO:9 and SEQ ID NO:6.

For example, the antibody or antigen-binding fragment thereof of the first aspect comprises a heavy chain variable domain and a light chain variable domain selected from the group consisting of the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
f) a heavy chain variable domain comprising SEQ ID NO:20 and a light chain variable domain comprising SEQ ID NO:23;
g) a heavy chain variable domain comprising SEQ ID NO:21 and a light chain variable domain comprising SEQ ID NO:24; and
h) a heavy chain variable domain comprising SEQ ID NO:22 and a light chain variable domain comprising SEQ ID NO:23,

for example from the group consisting of the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
f) a heavy chain variable domain comprising SEQ ID NO:20 and a light chain variable domain comprising SEQ ID NO:23; and
g) a heavy chain variable domain comprising SEQ ID NO:21 and a light chain variable domain comprising SEQ ID NO:24,

or from the group consisting of the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:20 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23; and
g) a heavy chain variable domain comprising SEQ ID NO:21 and a light chain variable domain comprising SEQ ID NO:24,

for example from the group consisting of the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23; and
f) a heavy chain variable domain comprising SEQ ID NO:20 and a light chain variable domain comprising SEQ ID NO:23,

for example from the group consisting of the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:20 and a light chain variable domain comprising SEQ ID NO:23;
d) a heavy chain variable domain comprising SEQ ID NO:18 and a light chain variable domain comprising SEQ ID NO:23; and
e) a heavy chain variable domain comprising SEQ ID NO:19 and a light chain variable domain comprising SEQ ID NO:23,

for example from the group consisting of the following VH/VL combinations:
a) a heavy chain variable domain comprising SEQ ID NO:18 and a light chain variable domain comprising SEQ ID NO:23;
b) a heavy chain variable domain comprising SEQ ID NO:15 and a light chain variable domain comprising SEQ ID NO:23,

for example an antibody or antigen-binding fragment thereof comprising the following VH/VL combination:
a) a heavy chain variable domain comprising SEQ ID NO:18 and a light chain variable domain comprising SEQ ID NO:23.

In another embodiment of the first aspect, the disclosure provides an antibody or antigen-binding fragment thereof, in which the amino acid sequence of VL-CDR1 is

(SEQ ID NO: 11)
KSSQSLLDSNGKTYLH.

In a more specific embodiment, the antibody or antigen-binding fragment thereof comprises a heavy chain variable domain and a light chain variable domain, wherein said heavy chain variable domain comprises the amino acid sequence SEQ ID NO:25 or an amino acid sequence having at least 80% identity to SEQ ID NO:25, provided that the three VH-CDR regions consist of SEQ ID NO:7, SEQ ID NO:8 and SEQ ID NO:3.

In another embodiment, the antibody or antigen-binding fragment thereof comprises a heavy chain variable domain and a light chain variable domain, wherein said light chain variable domain comprises the amino acid sequence SEQ ID NO:26 or an amino acid sequence having at least 80% identity to SEQ ID NO:26, provided that the three VL-CDR regions consist of SEQ ID NO:11, SEQ ID NO:9 and SEQ ID NO:6.

In yet another embodiment, the antibody or antigen-binding fragment thereof comprises both
a heavy chain variable domain comprising the amino acid sequence SEQ ID NO:25 or an amino acid sequence having at least 80% identity to SEQ ID NO:25, provided that the three VH-CDR regions consist of SEQ ID NO:7, SEQ ID NO:8 and SEQ ID NO:3, and
a light chain variable domain comprising the amino acid sequence SEQ ID NO:26 or an amino acid sequence having at least 80% identity to SEQ ID NO:26, provided that the three VL-CDR regions consist of SEQ ID NO:11, SEQ ID NO:9 and SEQ ID NO:6.

In another embodiment of the first aspect, the disclosure provides an antibody or antigen-binding fragment thereof, wherein the VH-CDR1, VH-CDR2, VL-CDR1 and VL-CDR2 regions consist of the following amino acid sequences:

VH-CDR1:
(SEQ ID NO: 12)
GYTLN;
VH-CDR2:
(SEQ ID NO: 13)
LINPYNGITTYNPKFMG;
VL-CDR1:
(SEQ ID NO: 11)
KSSQSLLDSNGKTYLH
VL-CDR2:
(SEQ ID NO: 14)
LVSKLDS.

In a more specific embodiment, the antibody or antigen-binding fragment thereof comprises a heavy chain variable domain and a light chain variable domain, wherein said heavy chain variable domain comprises the amino acid sequence SEQ ID NO:27 or an amino acid sequence having at least 80% identity to SEQ ID NO:27, provided that the three VH-CDR regions consist of SEQ ID NO:12, SEQ ID NO:13 and SEQ ID NO:3.

In another embodiment, the antibody or antigen-binding fragment thereof comprises a heavy chain variable domain and a light chain variable domain, wherein said light chain variable domain comprises the amino acid sequence SEQ ID NO:28 or an amino acid sequence having at least 80% identity to SEQ ID NO:28, provided that the three VL-CDR regions consist of SEQ ID NO:11, SEQ ID NO:14 and SEQ ID NO:6.

In yet another embodiment, the antibody or antigen-binding fragment thereof comprises both
a heavy chain variable domain comprising the amino acid sequence SEQ ID NO:27 or an amino acid sequence having at least 80% identity to SEQ ID NO:27, provided that the three VH-CDR regions consist of SEQ ID NO:12, SEQ ID NO:13 and SEQ ID NO:3, and
a light chain variable domain comprising the amino acid sequence SEQ ID NO:28 or an amino acid sequence having at least 80% identity to SEQ ID NO:28, provided that the three VL-CDR regions consist of SEQ ID NO:11, SEQ ID NO:14 and SEQ ID NO:6.

In certain embodiments, the definitions of VH and VL sequences of the antibody or antigen-binding fragment thereof is limited to any one of the listed sequences and sequences having at least 85%, such as at least 90%, such as at least 95%, such as at least 98%, such as at least 100% identity thereto.

In specific embodiments, the combinations of VH/VL are those present in the antibodies exemplified in Examples 1-13 (see Tables 1, 6 and 12 in particular).

For embodiments wherein the variable domains of the antibodies or antigen binding fragments are defined by a particular percentage sequence identity to a reference sequence, the VH and/or VL domains retain identical CDR sequences to those present in the reference sequence such that the variation is present only within the framework regions.

In one embodiment of the antibody or antigen binding fragment thereof according to the first aspect, the CDR regions are as defined using the Kabat convention, which is well known to a person of skill in the art of antibody technology (see e.g. Kabat (1991), Sequences of Proteins of Immunological Interest, 5^{th} edition, NIH Publication no 91-3242 from the US Department of Health and Human Services).

As a person of skill in the art is aware, Aβ peptides exist in various lengths and forms. Of particular relevance to the present disclosure is the N-terminus of the Aβ peptide, in that the antibodies herein bind to Aβ peptides having a truncation of two amino acid residues in comparison to full-length Aβ(i.e. Aβ peptides starting at the aspartic acid residue commonly denoted “D1”). On the other hand, the C-terminus of the peptide has little relevance to the present disclosure, and, as is well known, Aβ peptides may be as long as 43 amino acid residues or end already at e.g. position 28. In the terminology used herein, “AβpE3” is intended to encompass any Aβ peptide that begins with a pyroglutamate (“pE3”) residue corresponding to the third amino acid residue in the full-length peptide, regardless of C-terminus, which may or may not be truncated with respect to the full-length sequence. Likewise, “Aβ1-X” is used interchangeably with “full-length Aβ” and refers to any Aβ peptide that begins with the first amino acid residue (i.e. D1), regardless of its C-terminus.

A person of skill in the art is also aware that Aβ peptides may exist in various forms along the progressive aggregation thereof from monomers to insoluble plaques. Of particular relevance to the present disclosure, soluble forms of Aβ peptides may be present in monomer form, or in various oligomeric or further aggregated forms. Soluble forms of polymerized or aggregated Aβ peptides are collectively referred to as “protofibrils” in the present disclosure. For clarity, “protofibrils” is intended to encompass oligomers and higher order aggregates, but excludes insoluble fibrils or amyloid plaques.

In certain embodiments, an antibody or antigen-binding fragment thereof of the first aspect has an affinity for AβpE3 in a form selected from the group consisting of monomers, protofibrils, fibrils and plaques. In a more specific embodiment, the antibody or antigen-binding fragment thereof has an affinity for AβpE3 in a form selected from monomers and protofibrils. In yet another embodiment, the antibody or antigen-binding fragment thereof has an affinity for AβpE3 monomers. In a further embodiment, the antibody or antigen-binding fragment thereof has an affinity for AβpE3 protofibrils. It is noted that an antibody or antigen-binding fragment thereof according to the first aspect may have an affinity for AβpE3 generally, e.g. having an affinity for AβpE3 monomers as well as to AβpE3 protofibrils and/or other AβpE3 species.

Alternatively, the antibody or antigen-binding fragment thereof may exhibit a preference or selectivity for one form of AβpE3 over another. In one such embodiment, the antibody or antigen-binding fragment thereof has a higher binding affinity for protofibrils comprising AβpE3 than for AβpE3 monomers. Without wishing to be bound by theory, such higher affinity for protofibrils in embodiments of the antibody or fragment thereof may be due to avidity effects, insofar as the protofibril form of Aβ is thought to present a plurality of epitopes for the antibody to bind, in comparison to the monomeric form. As such, the affinity of the antibody for protofibrils may be measured and reported herein as an “apparent affinity” in a manner known to the skilled person. In one embodiment, the antibody or antigen-binding fragment thereof has at least 2× higher binding affinity for protofibrils comprising AβpE3 than for AβpE3 monomers, such as at least 10× higher, such as at least 40× higher, such as at least 100× higher, such as at least 200× higher binding affinity.

In certain embodiments, the antibodies and antigen-binding fragments thereof of the first aspect bind selectively to AβpE3. As used herein, the term “bind selectively” refers to the preferential binding of the antibody or antigen-binding fragment thereof to the AβpE3 target. In certain embodiments, the antibodies and antigen-binding fragments thereof of the first aspect do not bind to any appreciable extent to non-truncated amyloid beta, Aβ1-X. Put slightly differently, in one such embodiment, the antibody or antigen-binding fragment thereof has a higher binding affinity for AβpE3 monomers than for Aβ1-X monomers. In more specific embodiments, the antibody or antigen-binding fragment thereof has at least 2× higher binding affinity for AβpE3 monomers than for Aβ1-X monomers, such as at least 10× higher, such as at least 100× higher, such as at least 1000× higher, such as at least 3000× higher binding affinity.

As used herein, the terms “specific binding to X”, “selective binding to X” and “affinity for X”, wherein X is an antigen or an epitope, refer to a property of an antibody or antigen-binding fragment thereof which may be tested for example by ELISA, by surface plasmon resonance (SPR), by Kinetic Exclusion Assay (KinExA®) or by bio-layer interferometry (BLI). The skilled person is aware of these methods and others.

For example, binding affinity for antigen or epitope X may be tested in an experiment in which an antibody or antigen-binding fragment thereof to be tested is captured on ELISA plates coated with antigen X or an antigen exhibiting the epitope X, and a biotinylated detector antibody is added, followed by streptavidin-conjugated horse radish peroxidase (HRP). Alternatively, said detector antibody may be directly conjugated with HRP. Tetramethylbenzidine (TMB) substrate is added and the absorbance at 450 nm is measured using an ELISA multi-well plate reader. The skilled person may then interpret the results obtained by such experiments to establish at least a qualitative measure of the binding affinity of the antibody or antigen-binding fragment thereof for X. If a quantitative measure is desired, for example to determine the EC50 value (the half maximal effective concentration) for the interaction, ELISA may also be used. The response of the antibody or antigen-binding fragment thereof against a dilution series of X may be measured using ELISA as described above. The skilled person may then interpret the results obtained by such experiments and EC50 values may be calculated from the results, using for example GraphPad Prism v.9 and non-linear regression.

As used herein, the term “EC50” refers to the half maximal effective concentration of an antibody or antigen-binding fragment thereof which induces a response halfway between the baseline and maximum after a specified exposure time.

Additionally, inhibition ELISA may be used to obtain a quantitative measure of interaction by determination of the “IC50” (the half maximal inhibitory concentration). In an inhibition ELISA, the concentration of an antigen or epitope X in a fluid sample is measured by detecting interference in an expected signal output. In principle, a known antigen or epitope-bearing substance is used to coat a multi-well plate. In parallel, an antibody or antigen-binding fragment thereof with putative affinity for the antigen or epitope is added and incubated with a solution containing antigen at varied concentrations. Following standard blocking and washing steps, samples containing the mixture of said antibody or antigen-binding fragment thereof and the antigen or epitope are added to the well. Labeled detection antibody with affinity for the antigen- or epitope-binding antibody or antigen-binding fragment thereof is then applied for detection using relevant substrates (for example TMB). In principle, if there is a high concentration of antigen or epitope in the fluid sample, a significant reduction in signal output will be observed. In contrast, if there is very little antigen or epitope in the fluid sample, there will be very little reduction in the expected signal output. The skilled person appreciates that the signal output is also dependent on the affinity of the antibody or antigen-binding fragment thereof for said antigen or epitope.

As used herein, the term “IC50” refers to the half maximal inhibitory concentration of an antibody or antigen-binding fragment thereof which induces a response halfway between the baseline and maximum inhibition after a specified exposure time. Herein, a lower IC50 value indicates that a lower concentration of antigen or epitope is required to interfere with the binding of the detection antibody to the known antigen or epitope coated on the plate, as compared to a higher IC50 value. Thus, a lower IC50 value typically corresponds to a higher affinity.

The binding affinity of an antibody or antigen-binding fragment thereof may also be tested by SPR. For example, said binding affinity may be tested in an experiment in which antigen or epitope X is immobilized on a sensor chip of the instrument, and the sample containing the antibody or antigen-binding fragment thereof to be tested is passed over the chip. Alternatively, the antibody or antigen-binding fragment thereof to be tested may be immobilized on a sensor chip of the instrument, and a sample containing X is passed over the chip. The skilled person may then interpret the results obtained by such experiments to establish at least a qualitative measure of the binding affinity of the moiety for X. If a quantitative measure is desired, for example to determine a K_{D} value for the interaction, SPR may also be used. Binding values may for example be defined in a Biacore (Cytiva) or ProteOn XPR 36 (Bio-Rad) instrument. The antigen or epitope is suitably immobilized on a sensor chip of the instrument, and samples of the antibody or antigen-binding fragment thereof whose affinity is to be determined are prepared by serial dilution and injected. K_{D} values may then be calculated from the results using for example the 1:1 Langmuir binding model of the Biacore Insight Evaluation Software 2.0 or other suitable software, typically provided by the instrument manufacturer.

Another method for determining binding affinity of an antibody or antigen-binding fragment thereof to antigen or epitope X is the Kinetic Exclusion Assay (KinExA; Sapidyne Instruments Inc; Darling and Brault, Assay and Drug Dev Tech (2004) 2(6):647-657) for measurements of the equilibrium binding affinity and kinetics between unmodified molecules in solution. A KinExA Ko analysis requires immobilization of one interaction partner (e.g. the titrated binding partner) to a solid phase, which is then used as a probe to capture the other interaction partner (e.g. the constant binding partner) free in solution once an equilibrium is reached.

The binding affinity may also be measured by bio-layer interferometry (BLI), a label-free technology for measuring biomolecular interactions within the interactome. It is an optical analytical technique that analyzes the interference pattern of white light reflected from two surfaces: a layer of immobilized protein on the biosensor tip, and an internal reference layer. The binding between a ligand (antigen or epitope X) immobilized on the biosensor tip surface and an analyte (such as an antibody or antigen-binding fragment thereof with affinity for X) in solution produces an increase in optical thickness at the biosensor tip resulting in a wavelength shift, Δλ, which is a direct measure of the change in thickness of the biological layer. Interactions are measured in real time, providing the ability to monitor binding specificity, rates of association and dissociation, or concentration, with precision and accuracy.

The skilled person is aware of the above mentioned and other methods for measuring the affinity of an antibody or antigen-binding fragment thereof for antigen or epitope X, either qualitatively or quantitatively or both.

In one embodiment of the antibody or antigen-binding fragment thereof, it has a binding affinity (or an apparent binding affinity) for protofibrils comprising AβpE3 that corresponds to a K_{D} value of no more than 1 nM, such as between 1 and 200 pM, such as between 10 and 100 pM, as determined by SPR.

In another embodiment, the antibody or antigen-binding fragment thereof has a binding affinity for AβpE3 monomers that corresponds to a Ko value of no more than 100 nM, such as between 0.1 and 50 nM, such as between 0.5 and 10 nM, as determined by SPR.

In some embodiments of the antibody or antigen-binding fragment thereof according to the first aspect, the antibody or antigen-binding fragment thereof is selected from the group consisting of full-length antibodies, Fab fragments, Fab′ fragments, F(ab′)₂ fragments, Fv fragments, single chain Fv fragments, (scFv)₂ and domain antibodies. In one embodiment, said at least one antibody or antigen-binding fragment thereof is selected from full-length antibodies, Fab fragments and scFv fragments. In one particular embodiment, the antibody is a full-length antibody.

In one embodiment, the antibody or antigen-binding fragment thereof is of IgG class. In a more specific embodiment, the antibody or antigen-binding fragment thereof is of a sub-class selected from IgG1 and IgG4.

In one embodiment, the antibody or antigen-binding fragment thereof is monoclonal.

In one embodiment, the antibody or antigen-binding fragment thereof is selected from the group consisting of human antibodies, humanized antibodies, antibodies that have been mutated to reduce the antigenicity thereof in humans and antigen-binding fragments thereof.

As used herein, the term “antibody or antigen-binding fragment thereof” encompasses not only full-length or intact polyclonal or monoclonal antibodies, but also antigen-binding fragments thereof, such as Fab, Fab′, F(ab′)₂, Fab₃, Fv and variants thereof, fusion proteins comprising one or more antibody portions, humanized antibodies, chimeric antibodies, minibodies, diabodies, triabodies, tetrabodies, linear antibodies, single chain antibodies, multispecific antibodies (e.g. bispecific antibodies) and any other modified configuration of the immunoglobulin molecule that comprises an antigen recognition site of the required specificity, including glycosylation variants of antibodies, amino acid sequence variants of antibodies and covalently modified antibodies. Further examples of modified antibodies and antigen-binding fragments thereof include nanobodies, AlbudAbs, DARTs (dual affinity re-targeting), BiTEs (bispecific T-cell engager), TandAbs (tandem diabodies), DAFs (dual acting Fab), two-in-one antibodies, SMIPs (small modular immunopharmaceuticals), FynomAbs (fynomers fused to antibodies), DVD-Igs (dual variable domain immunoglobulin), CovX-bodies (peptide modified antibodies), duobodies and triomAbs. This listing of variants of antibodies and antigen-binding fragments thereof is not to be seen as limiting, and the skilled person is aware of other suitable variants.

A full-length antibody comprises two heavy chains and two light chains. Each heavy chain contains a heavy chain variable region (VH) and first, second and third constant regions (CH1, CH2 and CH3). Each light chain contains a light chain variable region (VL) and a light chain constant region (CL). Depending on the amino acid sequence of the constant domain of its heavy chains, antibodies are assigned to different classes. There are six major classes of antibodies: IgA, IgD, IgE, IgG, IgM and IgY, and several of these may be further divided into subclasses, e.g., IgG1, IgG2, IgG3, IgG4, IgA1 and IgA2. The term “full-length antibody” as used herein, refers to an antibody of any class, such as IgD, IgE, IgG, IgA, IgM or IgY (or any sub-class thereof). The subunit structures and three-dimensional configurations of different classes of antibodies are well known.

The term “antigen-binding fragment” refers to a portion or region of an antibody molecule, or a derivative thereof, that retains all or a significant part of the antigen binding of the corresponding full-length antibody. An antigen-binding fragment may comprise the heavy chain variable region (VH), the light chain variable region (VL), or both. Each of the VH and VL regions or domains typically contains three CDRs, i.e. CDR1, CDR2 and CDR3, denoted VH-CDR1, VH-CDR2 and VH-CDR3 for the CDRs from the VH domain and VL-CDR1, VL-CDR2 and VL-CDR3 for the CDRs from the VL domain. The three CDRs in VH or VL are flanked by framework regions (FR1, FR2, FR3 and FR4). As briefly listed above, examples of antigen-binding fragments include, but are not limited to: (1) a Fab fragment, which is a monovalent fragment having a VL-CL chain and a VH-CH1 chain; (2) a Fab′ fragment, which is a Fab fragment with the heavy chain hinge region, (3) a F(ab′)₂ fragment, which is a dimer of Fab′ fragments joined by the heavy chain hinge region, for example linked by a disulfide bridge at the hinge region; (4) an Fc fragment; (5) an Fv fragment, which is the minimum antibody fragment having the VL and VH domains of a single arm of an antibody; (6) a single chain Fv (scFv) fragment, which is a single polypeptide chain in which the VH and VL domains of an scFv are linked by a peptide linker; (7) an (scFv)₂, which comprises two VH domains and two VL domains, which are associated through the two VH domains via disulfide bridges and (8) a domain antibody, which may be an antibody single variable domain (VH or VL) polypeptide that specifically bind antigen. Antigen-binding fragments can be prepared via routine methods. For example, F(ab′)₂ fragments can be produced by pepsin digestion of a full-length antibody molecule, and Fab fragments can be generated by reducing the disulfide bridges of F(ab′)₂ fragments. Alternatively, portions can be prepared via recombinant technology by expressing the heavy and light chain portions in suitable host cells (e.g., E. coli, yeast, mammalian, plant or insect cells) and having them assembled to form the desired antigen-binding fragments either in vivo or in vitro. A single-chain antibody can be prepared via recombinant technology by linking a nucleotide sequence coding for a heavy chain variable region and a nucleotide sequence coding for a light chain variable region. For example, a flexible linker may be incorporated between the two variable regions.

Furthermore, the skilled person is aware of the meaning of the terms polyclonal antibodies and monoclonal antibodies. Polyclonal antibodies are normally generated by administering an antigen to an animal. Said antigen will evoke an immune response giving rise to polyclonal antibodies. Monoclonal antibodies are made by immunizing an animal, usually a mouse, with an antigen and the subsequent isolation of the spleen from said animal. Isolated spleen cells are immortalized by fusion with myeloma cells to give rise to hybridoma cells. Each hybridoma cell produces a unique monoclonal antibody.

The term “human antibody” as used herein, refers to antibodies having variable and constant regions corresponding to, or derived from, antibodies obtained from human subjects. The term “chimeric antibodies” as used herein, refers to recombinant or genetically engineered antibodies, such as for example antibodies with variable regions (VH and VL) of mouse origin and human constant region (Fc), to reduce the antibodies' immunogenicity. The term “humanized antibodies” refers to antibodies from non-human species whose protein sequences have been modified to increase their similarity to antibody variants produced naturally in humans, in order to reduce immunogenicity of the full antibody itself.

### Pharmaceutical Compositions

In a second aspect, there is provided a pharmaceutical composition comprising an antibody or antigen-binding fragment thereof as described herein and at least one pharmaceutically acceptable excipient or carrier.

Techniques for formulating antibodies for human therapeutic use are well known in the art and are reviewed, for example, in Wang et al. (2007), J Pharm Sci, 96:1-26, the contents of which are incorporated herein in their entirety.

Pharmaceutically acceptable excipients that may be used to formulate the compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene block polymers, polyethylene glycol and wool fat.

In certain embodiments, the pharmaceutical compositions are formulated for administration to a subject via any suitable route of administration including but not limited to intramuscular, intravenous, intradermal, intraperitoneal injection, subcutaneous, epidural, nasal, oral, rectal, topical, inhalational, buccal (e.g., sublingual) and transdermal administration. In preferred embodiments, the composition is formulated for intravenous or subcutaneous administration.

### Methods of Prevention, Treatment, Diagnosis, Prognosis and Detection

The antibodies or antigen-binding fragments thereof according to the present disclosure may be useful as therapeutic and/or diagnostic agents.

Hence, in a further aspect of the disclosure, there is provided an antibody or antigen-binding fragment thereof according to the first aspect, or a pharmaceutical composition according to the second aspect, for use as a medicament.

In yet another aspect of the disclosure, there is provided an antibody or antigen-binding fragment thereof according to the first aspect, or a pharmaceutical composition according to the second aspect, for use as a diagnostic agent.

Also provided are methods of preventing, treating or diagnosing disease or assessing disease prognosis, wherein an antibody or antigen-binding fragment thereof as disclosed herein is administered to a subject, typically a human subject.

Also provided is the use of the disclosed antibodies or antigen-binding fragments thereof for the manufacture of compositions (such as medicaments) for use in the prevention, treatment, diagnosis and/or prognosis of any one of the listed diseases.

Also provided are methods of detecting or diagnosing a disease in a subject, wherein the methods comprise contacting a sample obtained from the subject with an antibody or antigen-binding fragment thereof as described herein. These methods are typically in vitro methods.

Thus, said antibody or antigen-binding fragment thereof, or pharmaceutical composition comprising it, is useful in the treatment, prevention and/or diagnosis of a condition selected from neurological disorders or conditions characterized by accumulation and/or aggregation of Aβ, such as formation of amyloid plaques. Such diseases or conditions include but are not limited to Alzheimer's disease (AD) (including familial AD and sporadic AD), mild cognitive impairment (MCI), Lewy body dementia, neurodegeneration in Down's syndrome, cerebral amyloid angiopathy (CAA), hereditary cerebral hemorrhage with amyloidosis (Dutch type); as well as other diseases which are based on or associated with amylogenic proteins, such as progressive supranuclear palsy, multiple sclerosis, Creutzfeld-Jacob disease, cerebral amyloid angiopathy, Parkinson's disease, amyotrophic lateral sclerosis, cataract due to Aβ deposition, traumatic brain injury with an accumulation of Aβ, adult onset diabetes, senile cardiac amyloidosis and macular degeneration.

Thus, in one embodiment, there is provided an antibody or antigen-binding fragment thereof, or pharmaceutical composition comprising it, for use in the treatment, prevention, diagnosis and/or prognosis of an Aβ peptide-associated condition. In one embodiment, there is provided an antibody or antigen-binding fragment thereof, or pharmaceutical composition comprising it, for use in the treatment, prevention, diagnosis and/or prognosis of an Aβ peptide-associated condition, selected from the group consisting of Alzheimer's disease (AD) (including familial AD and sporadic AD), mild cognitive impairment (MCI), Lewy body dementia, neurodegeneration in Down's syndrome, cerebral amyloid angiopathy (CAA), hereditary cerebral hemorrhage with amyloidosis (Dutch type), progressive supranuclear palsy, multiple sclerosis, Creutzfeld-Jacob disease, cerebral amyloid angiopathy, Parkinson's disease, amyotrophic lateral sclerosis, cataract due to Aβ deposition, traumatic brain injury with an accumulation of Aβ, adult onset diabetes, senile cardiac amyloidosis and macular degeneration.

In one specific embodiment, said antibody or antigen-binding fragment thereof, or pharmaceutical composition comprising it, is provided for use in the treatment, prevention, diagnosis and/or prognosis of Alzheimer's disease.

In another aspect, there is provided a method of treatment, prevention, diagnosis and/or prognosis of an Aβ peptide-associated condition in a mammal having, or being at risk of developing, said disorder, comprising administering to said mammal an amount, such as a therapeutically effective amount, of an antibody or antigen-binding fragment thereof, or pharmaceutical composition comprising it.

In one embodiment, said Aβ peptide-associated condition is, for example, selected from the group consisting of Alzheimer's disease (AD) (including familial AD and sporadic AD), mild cognitive impairment (MCI), Lewy body dementia, neurodegeneration in Down's syndrome, cerebral amyloid angiopathy (CAA), hereditary cerebral hemorrhage with amyloidosis (Dutch type), progressive supranuclear palsy, multiple sclerosis, Creutzfeld-Jacob disease, cerebral amyloid angiopathy, Parkinson's disease, amyotrophic lateral sclerosis, cataract due to Aβ deposition, traumatic brain injury with an accumulation of Aβ, adult onset diabetes, senile cardiac amyloidosis and macular degeneration. In a more specific embodiment, said Aβ peptide-associated condition is Alzheimer's disease.

With regard to therapeutic or preventive use of the disclosed antibody, or antigen-binding fragment thereof, for the treatment of neurodegenerative diseases, there are several putative mechanisms of action. Without wishing to be bound by theory, non-limiting and independently possible mechanisms of action are for example binding to AβpE3 monomers to prevent seeding of Aβ aggregation, binding to and removal of soluble neurotoxic AβpE3-containing aggregates (protofibrils) and/or clearance of AβpE3-containing amyloid plaques to alleviate amyloidosis and improve cognitive function.

With regard to diagnostic or prognostic use of the disclosed antibody, or antigen-binding fragment thereof, in neurodegenerative diseases, the putatively harmful AβpE3 species can be detected and measured in patients at risk of disease or showing signs of incipient disease. One such method is PET scan using a radio-labelled antibody of the disclosure. Another method for diagnosis and prognosis is biochemical analysis analyzing the levels of AβpE3 in blood, plasma, CSF and other fluids, using such methods as ELISA, Mesoscale Discovery (MSD) or Simoa.

### INCORPORATION BY REFERENCE

Various publications are cited in the present application, each of which is incorporated by reference herein in its entirety.

### BRIEF DESCRIPTION OF THE FIGURES

FIG. 1 shows the binding of the indicated hybridoma clones to monomeric Aβ1-42, Aβ2-42, Aβ3-42, AβpE3-42 and Aβ4-42 using ELISA, as described in Example 1.

FIG. 2 shows the binding of the indicated hybridoma clones to AβpE3-42 protofibrils using ELISA, as described in Example 1.

FIG. 3 shows inhibition-response curves of the indicated recombinant antibodies against monomeric AβpE3-40 using inhibition ELISA, as described in Example 3.

FIG. 4 shows the inhibition-response curves of the indicated recombinant antibodies against monomeric Aβ1-40 using inhibition ELISA, as described in Example 3.

FIG. 5 shows the inhibition-response curves of the indicated recombinant antibodies against AβpE11-40 using inhibition ELISA, as described in Example 3.

FIG. 6 shows the inhibition-response curves of the indicated recombinant antibodies against AβpE3-42 protofibrils (PF) using inhibition ELISA, as described in Example 3.

FIG. 7 shows the binding interactions for the indicated recombinant antibodies against AβpE3-40 monomers, measured by SPR as described in Example 3.

FIG. 8 shows the binding interactions for the indicated recombinant antibodies against AβpE3-42 protofibrils, measured by SPR as described in Example 3.

FIG. 9 is a diagram showing the depletion of AβpE3-40 levels in soluble AD brain extracts by the indicated recombinant antibodies using immunoprecipitation, as described in Example 4.

FIG. 10 is a diagram showing the depletion of AβpE3-42 levels in soluble AD brain extracts by the indicated recombinant antibodies using immunoprecipitation, as described in Example 4.

FIG. 11 shows the binding of the indicated recombinant antibodies to amyloid plaques in sections of human brain by immunohistochemistry, as described in Example 4.

FIG. 12 shows the binding of the indicated humanized antibodies to monomeric AβpE3-28, Aβ1-28, Aβ2-28, Aβ3-28, Aβ4-28, Aβ5-28 and AβpE11-28 using inhibition ELISA, as described in Example 6.

FIG. 13 shows the binding of the indicated humanized antibodies to AβpE3-40 and Aβ1-40 monomers and AβpE3-42 and Aβ1-42 protofibrils using inhibition ELISA, as described in Example 6.

FIG. 14 shows the binding interactions for the indicated humanized antibodies against AβpE3-40 monomers, measured by SPR as described in Example 6.

FIG. 15 shows the binding interactions for the indicated humanized antibodies against AβpE3-42 protofibrils, measured by SPR as described in Example 6.

FIG. 16 is a diagram showing the immunoprecipitation of AβpE3-x levels in soluble AD brain extracts by the indicated humanized antibodies using immunoprecipitation, as described in Example 7.

FIG. 17 shows the binding of the indicated humanized antibodies to amyloid plaques in sections of human brain by immunohistochemistry, as described in Example 7.

FIG. 18 shows the concentration-response effects of the indicated humanized antibodies on AβpE3 aggregation, as described in Example 8.

FIG. 19 shows the effects of the indicated humanized antibodies on Aβ plaque clearance in AD brain sections, as described in Example 8.

FIG. 20 shows the pharmacokinetic plasma concentration-time profile in mouse of the indicated murine and humanized antibodies, as described in Example 9.

FIG. 21 shows the pharmacokinetic plasma concentration-time profile in mouse of the indicated humanized antibodies, as described in Example 10.

FIG. 22 shows the binding interactions for the indicated humanized antibodies against AβpE3-40 monomers, measured by SPR as described in Example 11.

FIG. 23 shows the binding interactions for the indicated humanized antibodies against AβpE3-42 protofibrils, measured by SPR as described in Example 11.

FIG. 24 is a diagram showing the immunoprecipitation of AβpE3-x levels in soluble AD brain extracts by the indicated humanized antibodies using immunoprecipitation, as described in Example 12.

FIG. 25 shows the binding of the indicated humanized antibodies to amyloid plaques in sections of human brain by immunohistochemistry, as described in Example 12.

FIG. 26 shows the concentration-response effects of the indicated humanized antibodies on AβpE3 aggregation, as described in Example 13.

FIG. 27 shows the effects of the indicated humanized antibodies on Aβ plaque clearance in AD brain sections, as described in Example 13.

### EXAMPLES

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment, but that the invention will include all embodiments falling within the scope of the appended claims. The invention will be further illustrated by the following non-limiting Examples.

### Example 1

### Generation and Screening of Antibodies to AβpE3

This example describes the immunization of BALB/c mice and subsequent generation and screening of hybridoma cell lines.

### Materials and Methods

Immunoqen preparation: Protofibrils produced from the AβpE3-42 peptide were used as immunogen. Briefly, AβpE3-42 peptide (American Peptide) was dissolved in 10 mM NaOH, pH>11 at a concentration of 100 μM. Protofibrils were prepared by neutralizing the AβpE3-42 peptide to pH 7.4 by adding 1:1 of a 2×PBS buffer to a final concentration of 50 μM. The peptide was incubated for 20 min at 37° C. for protofibril formation and was purified from remaining monomers by HPLC on a Superdex 75 10/300 GL size-exclusion column using a mobile-phase of 1×PBS, 0.1% Tween-20, pH 7.4. Prior to injection, the protofibril reaction was centrifuged at 16000×g for 5 min at 4° C. Fibrillar material was pelleted, and the supernatant contained soluble AβpE3-42 protofibrils and monomers. The protofibrils were eluted in the void volume of the column and the void fraction was collected. Repeated injections and collections of the void peaks were carried out to achieve enough material for immunization. A total of 14 injections were made, and 44 ml were collected and concentrated on 50 MWCO Amicon filters (UFC805024, Millipore) to a final concentration of 20 μM. Briefly, the Amicon filter was pre-wetted in PBS, 0.005% Tween-20, pH 7.4 for 2 h prior to sample loading. Protofibrils were added and concentrated 8-fold by centrifugation at 3200×g. Purified protofibrils were stored at −80° C. until use.

Immunization: BALB/c mice at 8 weeks of age (n=3) were immunized with AβpE3-42 protofibrils prepared as described above. AβpE3-42 protofibrils at a concentration of approximately 20 μM (86 μg/ml) were in sterile PBS, pH 7.4, containing 0.1% Tween-20. For each immunization, 400 μl AβpE3-42 protofibrils (~34 μg) were mixed with 5 μl ISCOM adjuvant (12 μg) and administered subcutaneously, 200 μl on each side. Each mouse received three or four immunizations. Plasma samples were collected between two and three weeks after each immunization. Once antibodies reactive against AβpE3-40 with end-point titers of around 1/100000 were detected in plasma, one final booster injection (immunogen without ISCOM adjuvant) was given intraperitoneally. After three days, mice were sacrificed, and spleens collected.

Tissue collection: Blood was collected from the tail vein at all time-points, except at the time of sacrifice when blood was collected from the heart. For sacrifice, mice were anesthetized with 300 mg/kg ketamine and 4 mg/kg medetomidine and blood was sampled from the right atrium into Microtainer tubes followed by centrifugation of the samples at 2400×g for 10 min and transfer of plasma to pre-labeled low-binding Eppendorf tubes. Plasma samples were immediately frozen on dry ice and stored at −80° C. The spleens were collected by opening the abdominal cavity and dissecting out the intact spleens. The spleens were placed in a 15 ml test tube containing 5 ml DMEM media (2× PenStrep) at room temperature (RT) and transported on ice within an hour for preparation of a spleen cell suspension.

Plasma screening by direct ELISA: Plasma samples were analyzed by direct ELISA for reactivity against AβpE3-40 monomers after each immunization to determine when to stop immunizations and initiate hybridoma generation. The AβpE3-40 peptide (Anaspec) was dissolved to 100 μM in 10 mM NaOH+0.005% Tween-20 and stored in aliquots at −80° C. For coating, a 0.5 μM solution was prepared by diluting newly thawed 100 μM AβpE3-40 peptide in PBS, and an ELISA microtiter plate was coated with 50 μl AβpE3-per well overnight at +4° C. The plate was washed four times with 1×ELISA washing buffer (containing 0.28 mM NaH₂PO₄, 2.5 mM Na₂HPO₄, 150 mM NaCl, 0.1% Tween-20 and 0.0075% Kathon CG), followed by blocking of the residual binding capacity of the plate by adding 100 μl/well of Pierce blocking buffer and incubation for 1 h at RT with shaking (900 rpm). After discarding the blocking buffer, samples (mouse plasma) or standard was added to each well. Plasma samples from immunized mice were diluted at least 1:1000 and further diluted 1:2 in the wells in seven steps. Plasma from an un-immunized mouse was used as a negative control. The control antibody mAb6E10 (Covance #SIG-39320, epitope: amino acids 1-16 of Aβ) was used to make a standard curve by 2-fold serial dilution starting at 1 ng/ml (final concentration range of 0.016-1 ng/ml). The samples were incubated for 90 min at RT with shaking (900 rpm). The plate was washed as above, and 50 μl of HRP-conjugated anti-mouse IgG (diluted 1/10 000 in incubation buffer consisting of 1× Dulbecco's PBS with 0.1% BSA and 0.05% Tween-20) was added to each well, followed by incubation for 1 h at RT with shaking (900 rpm). For detection, the plate was once again washed as above, and 50 μl of room tempered TMB was added to each well and the plate was incubated at RT for 15 min without shaking. The reaction was stopped by adding 50 μl of 2 M H₂SO₄ and the plate was read at a wavelength of 450 nm within 15 min. An end point titer of 1/100000 was considered high enough, and after this had been reached, no more immunizations were performed.

Generation of hybridomas: Isolated splenocytes from sacrificed mice were fused with cells from an immortalized cell line (SP2/0) to generate hybridomas. Briefly, a single cell suspension of the spleen from an immunized mouse was prepared and mixed with SP2/0 cells at a 1:2 ratio. The cells were fused using polyethylene glycol and the cells were dispensed in 96 well cell culture plates. Media was changed at 7 days and 10-14 days post fusion. The wells/hybridomas were screened for reactivity against AβpE3-42 protofibrils using ELISA. Positive clones were diluted using limiting dilution assays to certify monoclonality. Clones of interest were cryopreserved, expanded for production of antibody, and sequenced.

Antigens used for hybridoma screening by ELISA: AβpE3-42 and Aβ1-42 monomers (American Peptide) and AβpE3-42 and Aβ1-42 protofibrils were used for screening of hybridoma clones. The protofibrils were generated using the respective monomers. Briefly, to generate AβpE3-42 protofibrils, the AβpE3-42 peptide was dissolved in 10 mM NaOH, 0.005% Tween-20, pH>11 at a concentration of 100 μM. Protofibrils were prepared by neutralization of the AβpE3-42 peptide to pH 7.4 by adding 1:1 of a 2×PBS buffer to a final concentration of 50 μM. The peptide was incubated for ~30 min at 37° C. for protofibril formation and purified from remaining monomers by HPLC on a Superdex 75 Increase 3.2/300 size-exclusion column using a mobile phase of 1×PBS, 0.1% Tween-20, pH 7.4. Prior to injection, the protofibril reaction was centrifuged at 16000×g for 5 min at 4° C. The void peak containing AβpE3-42 protofibrils was collected and the concentration was determined using SEC and a calibration curve of an Aβ protofibril standard with a known concentration. The same procedure was used to produce Aβ1-42 protofibrils from the corresponding monomers.

Hybridoma screening by ELISA: Supernatants from generated hybridomas were characterized using ELISA with Aβ1-42 or AβpE3-42 monomers or protofibrils as capture antigens. An ELISA microtiter plate was coated with a polyclonal rabbit anti-Aβ42 antibody (diluted to 0.5 μg/ml in PBS and 50 μl added per well) and incubated overnight at 4° C. or for 1 h at 37° C. The plate was washed 4 times with 1×ELISA washing buffer followed by blocking of the residual binding capacity of the plate by adding 100 μl/well of Pierce blocking buffer and incubating for 1 h at RT with shaking (900 rpm). The blocking buffer was discarded, and each antigen was added (diluted in incubation buffer to 5 nM and 50 μl added per well) and the plate was incubated for 1 h at RT with shaking (900 rpm). The plate was washed 4 times as described above, followed by addition of sample. Supernatants from hybridoma cell cultures were added undiluted or diluted 1:2 in incubation buffer to each well at a volume of 50 μl/well. The samples were incubated for 1 h at RT with shaking (900 rpm). Following a washing step, an HRP-conjugated anti-mouse IgG antibody (diluted 1/5000 in incubation buffer) was added to each well (50 μl/well) and the plate was incubated for 1 h at RT with shaking (900 rpm). After an additional washing step, detection was performed by adding 50 μl of room tempered TMB to each well, followed by incubation at RT in the dark for 10 min without shaking. The reaction was stopped by adding 50 μl of 2 M H₂SO₄ and the microtiter plate was read at a wavelength of 450 nm, preferably within 15 min. Positive clones with specific binding to AβpE3-42 were further characterized using Aβ1-42, Aβ2-42, Aβ3-42, AβpE3-42 and Aβ4-42 monomers (all purchased from Anaspec) as capture antigens in ELISA, according to the method described above. The supernatants were diluted 1/243 in ELISA regardless of antibody concentration, so measured OD₄₅₀ values did not necessarily correlate to binding affinity.

Antibody concentration determination: The antibody concentration in the respective hybridoma supernatants was measured using a standard sandwich ELISA. Microtiter plates were coated with an anti-mouse IgG antibody recognizing the F(ab′)₂ part of mouse IgG (0.5 μg/ml, 50 μl per well) overnight at 4° C. without shaking. The residual binding capacity of the plate was blocked by adding 200 μl/well of blocking buffer and incubating for 1 h at RT with shaking (900 rpm). The plate was washed three times with 1×ELISA washing buffer, followed by addition of the samples. Hybridoma supernatants were diluted 1/250 and added in duplicates to the ELISA plate (200 μl/well) and serially diluted 2-fold in seven steps to ensure that the sample dilution was within the range of the standard. The Aβ protofibril-selective mouse antibody mAb158 (Englund et al (2007), J Neurochem 103(1):334-45) was used to make a standard curve by 2-fold serial dilution to generate a final concentration range of 15-500 μg/ml). The plate was incubated for 2 h at RT with shaking (900 rpm). The plate was washed as above, and 50 μl of HRP-conjugated anti-mouse IgG antibody that was also F(ab)₂ specific (diluted 1/2500 in incubation buffer) was added to each well, followed by incubation for 1 h at RT with shaking (900 rpm). For detection, the plate was once again washed as above, and 100 μl of room tempered TMB was added to each well and the plate was incubated at RT for 5-20 min without shaking. The reaction was stopped by adding 50 μl of 2 M H₂SO₄ and the plate was read at a wavelength of 450 nm within 15 min.

### Results

Generation of Monoclonal Antibodies by Hybridoma Technology: Antibodies that bind selectively to AβpE3 were generated by immunization using AβpE3-42 protofibrils. The plasma samples were analyzed by ELISA for reactivity against the AβpE3-40 monomer. When titers were at least 1/100000 the mice were sacrificed, and the spleens were collected and used for hybridoma generation.

A total of 22 AβpE3-42 reactive hybridomas were generated. Their specificity for AβpE3-42 monomers and AβpE3-42 protofibrils, compared to for Aβ1-42 monomers and Aβ1-42 protofibrils, was tested by ELISA. Twelve of the clones bound to all four Aβ forms tested, whereas ten of the clones bound specifically to AβpE3-42. None of the clones were selective for protofibrils but bound equally well to AβpE3-42 monomers as to AβpE3-42 protofibrils.

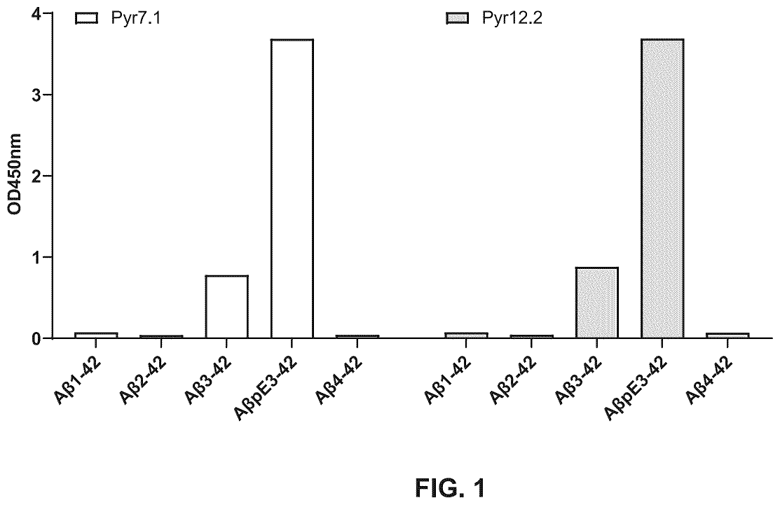

The ten clones that were specific binders to AβpE3-42 were further characterized using Aβ1-42, Aβ2-42, Aβ3-42, AβpE3-42 and Aβ4-42 monomers as capture reagent in ELISA. All ten clones tested were specific for AβpE3-42 monomers, with only some weak cross-reactivity to Aβ3-42 monomers. FIG. 1 shows the result of this experiment for two selected clones, denoted Pyr7.1 and Pyr12.2.

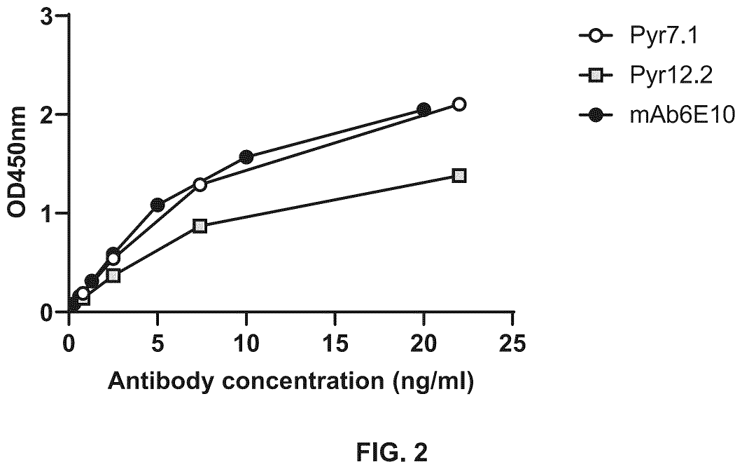

To compare the binding strength of the Pyr7.1 and Pyr12.2 antibody clones, antibody concentration was first determined, and the same amount of each clone was then loaded on an ELISA plate using AβpE3-42 protofibrils as capture. Both clones demonstrated binding to AβpE3-42 protofibrils, with binding comparable to the positive control mAb6E10 (FIG. 2).

### Example 2

### Hybridoma Sequencing and Production of Recombinant Antibodies

### Materials and Methods

Hybridoma sequencing: Hybridoma clones producing monoclonal antibodies, generated and characterized as described in Example 1 and having a demonstrated specificity for AβpE3-42 monomers and protofibrils, were sequenced by whole transcriptome shotgun sequencing. The DNA and protein sequences of mature VH and VL regions were identified.

Expression, production and purification: The variable domains were designed and optimized for expression in mammalian cells (HEK293) prior to being synthesized. The sequences were then subcloned into a cloning and expression vector (Absolute Antibody) for the appropriate isotype and subtype of immunoglobulin heavy and light chains. HEK293 cells were passaged to the optimum stage for transient transfection. Cells were transiently transfected with heavy and light chain expression vectors and cultured for a further 6-14 days. Cultures were harvested and a one-step purification was performed using affinity chromatography, after which the purified antibodies were buffer exchanged into PBS. Antibodies were analyzed for purity by SDS-PAGE and the concentration determined by UV spectroscopy.

### Results

Hybridoma sequencing and recombinant antibody production: Hybridoma clones with a demonstrated specificity for AβpE3-42 monomers and protofibrils were sequenced, and the sequences of selected antibodies Pyr7.1 and Pyr12.2 are reported.

The amino acid sequences of the entire antibodies were obtained. Amino acid sequences for the respective variable heavy (VH) and variable light (VL) chains are given in Table 1 below, as well as the sequences of the constant regions shared by all antibodies. Complementarity determining regions (CDRs) were identified using the Kabat definition.

TABLE 1
Amino acid sequences of selected monoclonal antibodies

| | | | SEQ |
|---|---|---|---|
| | | | ID |
| Antibody | Region | Amino acid sequence | NO: |
| Pyr7.1 | Heavy chain | | |
| | VH | KVQLQQSGPELVKPGTSIKMSCKTSG | 27 |
| | | YSFTGYTLNWVKQSPGKNPEWIGLIN | |
| | | PYNGITTYNPKFMGKATLTVDKSSSTA | |
| | | YMELLSLTSEDSAVYYCSREGNWEGV | |
| | | YWGQGTLVTVSA | |
| | VH-CDR1 | GYTLN | 12 |
| | VH-CDR2 | LINPYNGITTYNPKFMG | 13 |
| | VH-CDR3 | EGNWEGVY | 3 |
| | Light chain | | |
| | VL | DVVMTQTPLTLSVTIGQPASISCKSSQ | 28 |
| | | SLLDSNGKTYLHWLLLRPGQSPKRLIY | |
| | | LVSKLDSGVPDRFTGSGSGTDFTLKIS | |
| | | RVEAEDLGVYFCVQGTHFPFTFGSGT | |
| | | KLEIK | |
| | VL-CDR1 | KSSQSLLDSNGKTYLH | 11 |
| | VL-CDR2 | LVSKLDS | 14 |
| | VL-CDR3 | VQGTHFPFT | 6 |
| Pyr12.2 | Heavy chain | | |
| | VH | EVQLQQSGPELVKPGTSIKMSCKASG | 25 |
| | | YSFTGFTMNWVKQSHGKNLEWIGLIN | |
| | | PYNGVTTYNQKFKGKATITVDKSSRTA | |
| | | YMELLSLTYEDSAVYYCTREGNWEGV | |
| | | YWGQGTPVTVSA | |
| | VH-CDR1 | GFTMN | 7 |
| | VH-CDR2 | LINPYNGVTTYNQKFKG | 8 |
| | VH-CDR3 | EGNWEGVY | 3 |
| | Light chain | | |
| | VL | EVVLTQTPLTLSVTIGQSASISCKSSQS | 26 |
| | | LLDSNGKTYLHWFLLRPGQSPKRLIYL | |
| | | VSILDSGVPDRFTGSGSGTDFTLKISR | |
| | | VEAEDLGIYYCVQGTHFPFTFGSGTKL | |
| | | EIK | |
| | VL-CDR1 | KSSQSLLDSNGKTYLH | 11 |
| | VL-CDR2 | LVSILDS | 9 |
| | VL-CDR3 | VQGTHFPFT | 6 |
| Murine | Constant | AKTTAPSVYPLAPVCGGTTGSSVTLG | 29 |
| IgG2c | heavy chain | CLVKGYFPEPVTLTWNSGSLSSGVHT | |
| constant | region | FPALLQSGLYTLSSSVTVTSNTWPSQT | |
| region | | ITCNVAHPASSTKVDKKIESRRPIPPNS | |
| | | CPPCKECSIFPAPDLLGGPSVFIFPPKI | |
| | | KDVLMISLSPIVTCVVVDVSEDDPDVQI | |
| | | SWFVNNVEVHTAQTQTHREDYNSTLR | |
| | | VVSALPIQHQDWMSGKEFKCKVNNRA | |
| | | LPSPIEKTISKPRGPVRAPQVYVLPPPA | |
| | | EEMTKKEFSLTCMITDFLPAEIAVDWT | |
| | | SNGHKELNYKNTAPVLDTDGSYFMYS | |
| | | KLRVQKSTWEKGSLFACSVVHEGLHN | |
| | | HHTTKTISRSLGK | |
| Murine | Constant | RADAAPTVSIFPPSSEQLTSGGASVVC | 30 |
| kappa | light | FLNNFYPKDINVKWKIDGSERQNGVLN | |
| constant | chain | SWTDQDSKDSTYSMSSTLTLTKDEYE | |
| region | region | RHNSYTCEATHKTSTSPIVKSFNRNEC | |

The monoclonal antibodies Pyr7.1 and Pyr12.2 were selected for production as recombinant IgG2c antibodies. All recombinant antibodies were successfully produced and purified to a final concentration of 1 mg/ml. Antibody purity, as defined by SEC-HPLC, was >98% monomer for all antibodies.

### Example 3

### Characterization of Recombinant Antibodies

This example describes the characterization of the affinity, selectivity and specificity of the recombinant antibodies produced in Example 2 by inhibition ELISA and SPR.

### Materials and Methods

Aβ monomer species and Aβ protofibrils: The following Aβ peptides were used for characterization of the binding of the recombinant antibodies to different Aβ species: AβpE3-40, Aβ1-40, Aβ1-28 and AβpE11-40, all purchased from Bachem, and Aβ2-28, Aβ3-28, Aβ4-28 and AβpE11-28, all custom-made by and purchased from Innovagen. Aβ peptides from Bachem were dissolved in 10 mM NaOH, 0.005% Tween-20, pH>11 at a concentration of 100 μM. Aβ peptides from Innovagen were dissolved in 1×PBS, pH 7.4 at a concentration of 300 μM. Aliquots were made and stored at −80° C. until analysis. All peptides were verified to be monomeric by size-exclusion chromatography.

Protofibrils were prepared using the AβpE3-42 peptide from Bachem. Briefly, the AβpE3-42 peptide was dissolved in 10 mM NaOH, 0.005% Tween-20, pH>11 at a concentration of 100 μM. Protofibrils were prepared by neutralizing the AβpE3-42 peptide to pH 7.4 by adding 1:1 of a 2×PBS buffer to a final concentration of 50 μM. The peptide was incubated for −30 min at 37° C. for protofibril formation and purified from remaining monomers by HPLC on a Superdex 75 Increase 3.2/300 size-exclusion column using a mobile-phase of 1×PBS, 0.1% Tween-20, pH 7.4. Prior to injection, the protofibril reaction was centrifuged at 16000×g for 5 min at 4° C. to remove insoluble fibrils. The void peak containing the AβpE3-42 protofibrils was collected and the concentration determined using SEC and a calibration curve of an Aβ protofibril standard with a known concentration.

Selectivity evaluation and IC50 determination by inhibition ELISA: The binding of selected recombinant antibodies Pyr7.1 and Pyr12.2 to different Aβ antigens (AβpE3-40, Aβ1-40 and AβpE11-40 monomers and AβpE3-42 protofibrils) was evaluated by inhibition ELISA. For binding to Aβ1-40 and AβpE11-40, the positive control 4G8 (Covance #SIG-39320, epitope: amino acids 17-24 of Aβ) was included. The recombinant antibodies were incubated at a fixed concentration (0.05 μg/ml) with titrating concentrations of the different Aβ antigens. After incubation for 45 min at 900 rpm to reach equilibrium, the antibody-Aβ samples were added to a blocked and washed ELISA plate with an AβpE3-40 coat (0.5 μM). The samples were incubated on the plate for 25 min without shaking followed by washing, incubation with detection antibody, another washing step and finally incubation with alkaline phosphatase substrate. Optical density at 405 nm was read, and the collected data was analyzed using a four-parameter variable slope non-linear fit to determine IC₅₀ values.

Affinity and specificity evaluation and Ko determination by surface plasmon resonance: Binding interactions between antigens and antibodies were evaluated by SPR using a Biacore 8K instrument (Cytiva) according to standard procedures. The binding of selected recombinant antibodies Pyr7.1 and Pyr12.2 to AβpE3-40 monomers and protofibrils and their selectivity against Aβ1-28 monomers was evaluated. Additionally, the specificity towards different N-truncated forms of Aβ (Aβ2-28, Aβ3-28, Aβ4-28 and AβpE11-28 monomers) was evaluated.

Single cycle kinetics with the antibodies immobilized on a CM5 chip was used to measure the binding of the antibodies to the different monomers (AβpE3-40, AβpE11-28, Aβ1-28, Aβ2-28, Aβ3-28 and Aβ4-28). For the measurements, 5 μg/ml of analyte antibody was immobilized on the chip. The AβpE3-40 monomer was then injected over the chip using a 2-fold dilution in five steps starting at 250 nM for AβpE3-40 with a dissociation time of 20 min and at 2500 nM for all other monomers with a dissociation time of 10 min. Regeneration of the surface between cycles was done by injecting 30 μl 3 M MgCl₂. The binding data was fitted to a 1:1 interaction model.

Single cycle kinetics was also used to measure binding of the antibodies to AβpE3-42 protofibrils. The AβpE3-42 protofibrils (138 ng/ml) were coupled to a CM5 chip using general Biacore coupling chemistry (immobilization low levels). For binding to AβpE3-42 protofibrils, the antibodies were injected over the chip using a 3-fold dilution series in five steps starting at 700 nM, using 2 min injection of every antibody concentration and a 60 min dissociation time. Regeneration of the surface between cycles was done by injecting 30 μl 10 mM glycine-HCl pH 1.7. The binding data was fitted to a 1:1 interaction model. The use of the 1:1 interaction model gives the Ko for the binding of the antibodies to Aβ monomers and an apparent Ko for the binding of the antibodies to Aβ protofibrils.

In all SPR experiments, 1×HBS-EP+(Cytiva, cat. no. BR100669) was used to dilute antibodies and target antigens. Experiments were performed at 25° C.

### Results

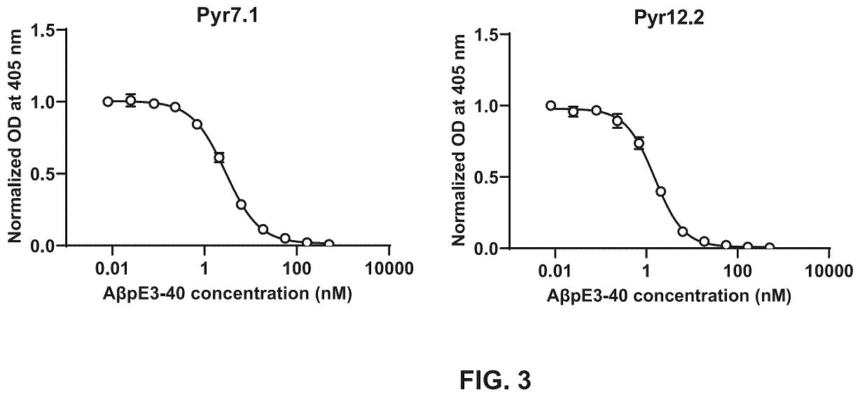

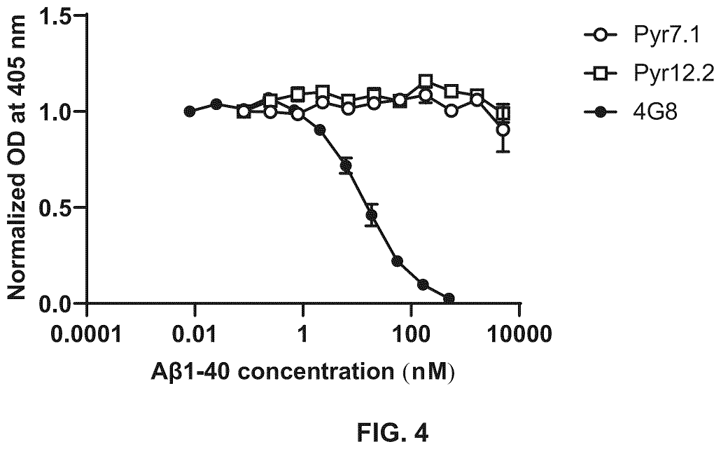

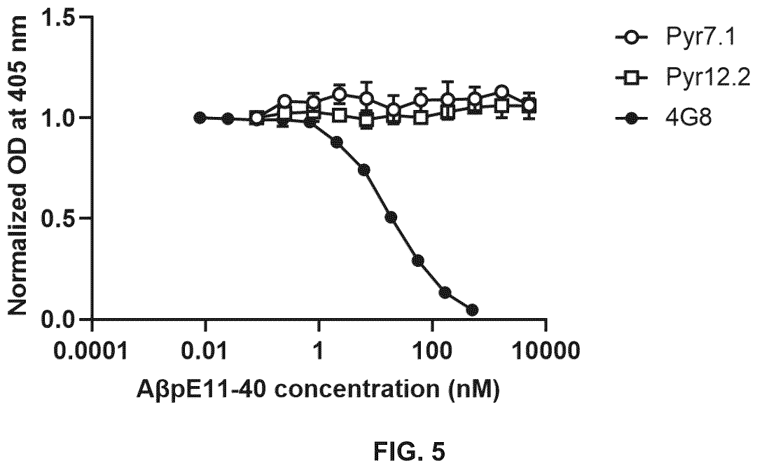

Selectivity evaluation and IC50 determination by inhibition ELISA: The binding of the recombinant antibodies to AβpE3-40 and selectivity versus Aβ1-40 and AβpE11-40 monomers was initially evaluated using inhibition ELISA. Recombinant antibodies Pyr7.1 and Pyr12.2 demonstrated binding to AβpE3-40 monomer in solution (FIG. 3). None of the antibodies bound Aβ1-40 in solution at concentrations up to 5 μM, suggesting the IC₅₀ values were >5 μM (FIG. 4). The positive control antibody 4G8 demonstrated binding to Aβ1-40, as expected. Recombinant antibodies Pyr7.1 and Pyr12.2 were also tested for binding to AβpE11-40 monomers using inhibition ELISA. Here, no binding was observed at antigen concentrations up to 5 μM, suggesting the IC₅₀ values were >5 μM (FIG. 5). The positive control antibody 4G8 demonstrated binding to AβpE11-40, as expected. The calculated IC₅₀ values are listed in Table 2.

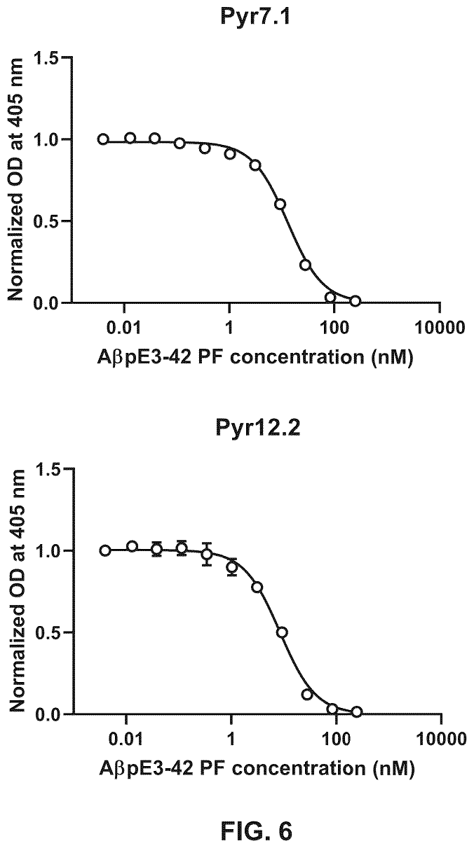

The binding of the recombinant antibodies to AβpE3-42 protofibrils was evaluated using inhibition ELISA. Both antibodies demonstrated binding to AβpE3-42 protofibrils in solution (FIG. 6). The calculated IC₅₀ values are listed in Table 2.

TABLE 2
Summary of results from inhibition ELISA

| | AβpE3-40 | Aβ1-40 | AβpE11-40 | AβpE3-42 |
|---|---|---|---|---|
| | monomer | monomer | monomer | protofibril |
| | IC₅₀ | IC₅₀ | IC₅₀ | IC₅₀ |
| Antibody | (nM) | (nM) | (nM) | (nM) |
| Pyr7.1 | 2.85 | >5000 | >5000 | 12.8 |
| Pyr12.2 | 1.52 | >5000 | >5000 | 8.43 |

Affinity and specificity evaluation and Ko determination by surface plasmon resonance: The recombinant antibodies Pyr7.1 and Pyr12.2 were evaluated by SPR for affinity and specificity and their K_{D} values were determined.

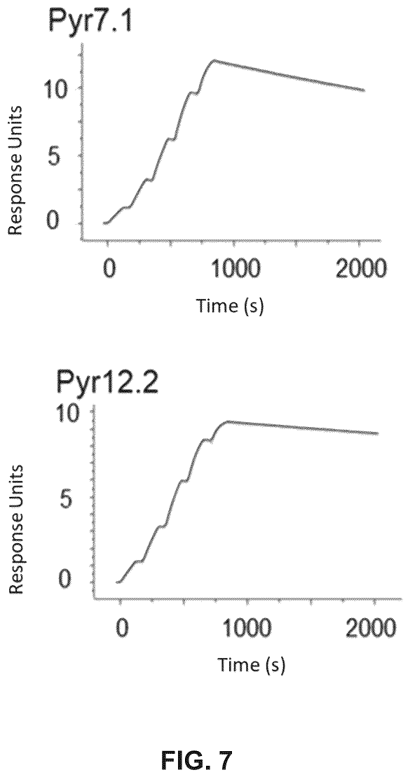

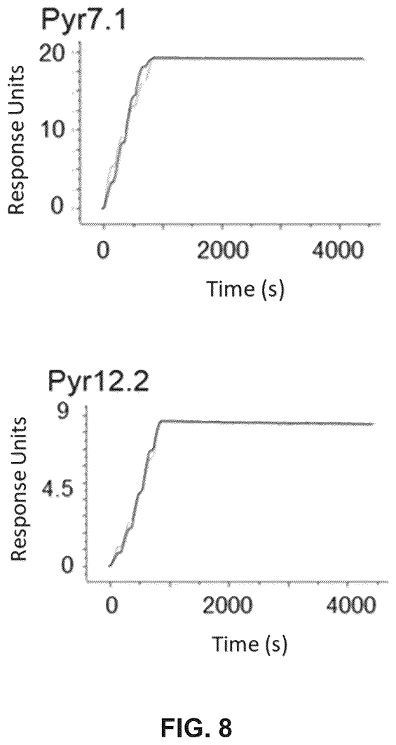

Both antibodies demonstrated binding to AβpE3-40 monomers and AβpE3-42 protofibrils. The apparent affinities of Pyr7.1 and Pyr12.2 for AβpE3-42 protofibrils were probably underestimated, because the k_{d} values for most of the binding experiments were outside the instrument's detection limits. All kinetic data from such binding experiments were excluded when calculating affinities. The calculated kₐ, k_{d} and (apparent) K_{D} values are shown in Table 3 and Table 4 below. Representative sensorgrams are shown in FIG. 7 and FIG. 8.

TABLE 3
Summary of SPR analysis of binding to AβpE3-40 monomers

| | AβpE3-40 monomer | | |
|---|---|---|---|
| | kₐ (M⁻¹s⁻¹) | k_{d} (s⁻¹) | K_{D} (nM) |
| Antibody | Mean ± SD | Mean ± SD | Mean ± SD |
| Pyr7.1 | 5.32 ± 0.11 e4 | 1.22 ± 0.20 e−4 | 2.30 ± 0.40 |
| Pyr12.2 | 7.18 ± 0.22 e4 | 4.12 ± 1.41 e−5 | 0.57 ± 0.19 |

TABLE 4
Summary of SPR analysis of binding to AβpE3-42 protofibrils

| | AβpE3-42 protofibril | | |
|---|---|---|---|
| | kₐ (M⁻¹s⁻¹) | k_{d} (s⁻¹) | K_{D} (nM) |
| Antibody | Mean ± SD | Mean ± SD | Mean ± SD |
| Pyr7.1 | 7.82 ± 1.48 e4 | 2.29 ± 0.89 e−6 | 31.0 ± 15.2 |
| Pyr12.2 | 6.31 ± 1.22 e4 | 3.31 ± 1.93 e−6 | 53.6 ± 31.0 |

The recombinant antibodies did not show any binding to Aβ1-28 up to 2500 nM of monomer.

Specificity of recombinant antibodies evaluated by surface plasmon resonance: The specificity of the recombinant antibodies to different N-truncated forms of Aβ monomers was evaluated by SPR. Pyr7.1 and Pyr12.2 had no binding to the Aβ32-28 monomer in the concentration range tested. Pyr12.2 bound the Aβ3-28 monomer with nM affinity, whereas no binding was detected for Pyr7.1. Both antibodies bound the Aβ4-28 monomer with μM affinity. No binding was observed for any of the antibodies to Aβ3pE11-28 up to a concentration of 2500 nM. The calculated kₐ, k_{d} and K_{D} values are shown in Table 5.

TABLE 5
Summary of SPR analysis of binding to Aβ2-28,
Aβ3-28, Aβ4-28 and AβpE11-28 monomers

| | kₐ (M⁻¹s⁻¹) | k_{d} (s⁻¹) | K_{D} (μM) |
|---|---|---|---|
| Antibody | Mean ± SD | Mean ± SD | Mean ± SD |
| | Aβ2-28 monomer | | |
| Pyr7.1 | — | — | No binding |
| Pyr12.2 | — | — | No binding |
| | Aβ3-28 monomer | | |
| Pyr7.1 | — | — | No binding |
| Pyr12.2 | 1.75 ± 0.94 e3 | 5.77 ± 1.22 e−5 | 42.6 ± 18.9 |
| | Aβ4-28 monomer | | |
| Pyr7.1 | 1.02 ± 0.81 e5 | 4.54 ± 2.55 e−1 | 8.31 ± 2.55 |
| Pyr12.2 | 7.82 ± 1.95 e4 | 4.81 ± 0.60 e−1 | 6.76 ± 2.62 |
| | AβpE11-28 monomer | | |
| Pyr7.1 | — | — | No binding |
| Pyr12.2 | — | — | No binding |

### Example 4

Target Binding of Recombinant Antibodies in Brain from Human Alzheimer's Disease Patients and Non-Demented Controls

This Example describes target binding of recombinant antibodies Pyr7.1 and Pyr12.2, produced as described in Example 2, as tested by immunoprecipitation on human brain extracts and immunohistochemistry on human brain sections from AD patients and NDE controls.

### Materials and Methods

Brain tissue homogenization and sample preparation: Fresh frozen human brain cortical tissue from Alzheimer's disease (AD) patients and non-demented (NDE) controls were homogenized in a Potter-Elvehjem homogenizer at 1:10 weight:volume in Tris-buffered saline (TBS) buffer followed by centrifugation at 16000×g for 1 h. The resulting supernatant was frozen at −80° C. until analysis.

Target binding in human Alzheimer's disease brain extracts by immunoprecipitation: Antibody binding to target in human AD brain was analyzed by immunodepletion, a method for removal of target protein in a sample using an antibody specific for the target molecule. Briefly, each of the recombinant antibodies Pyr7.1 and Pyr12.2, covalently coupled to magnetic Dynabeads, was incubated with soluble TBS brain extracts from AD and non-demented (NDE) control cases for 1 h at RT with rotation. The bead-bound target was separated by a magnet and depleted from the extracts. The depleted brain extracts (supernatants) were analyzed using an AβpE3-40 kit and an MSD assay for measurements of AβpE3-42 levels. Target binding by the recombinant antibodies was evaluated by the reduction of measured AβpE3-40 or AβpE3-42 levels in depleted samples compared to levels in non-depleted brain extracts. A complete target binding was demonstrated when levels in depleted brain extracts were below the lower limit of quantification (LLOQ) for the assay.

A sandwich ELISA kit from Immunobiological Laboratories (product code: 27418) was used to measure levels of AβpE3-40 in human AD brain. Each assay kit contains all necessary components including antibodies, standard calibrator and plates precoated with a human anti-Aβ mouse IgG monoclonal capture antibody (epitope at amino acid positions 35-40 of Aβ). Briefly, diluted standard calibrator and test samples were allowed to bind to the plate during incubation at 4° C. overnight. After a wash step, an HRP conjugated anti-human AβpE3 antibody (8E1, included in the kit) was added, followed by an incubation for 1 h at 4° C. After an additional wash step, TMB was added as a coloring agent (chromogen) and the plate was incubated for 30 min before the reaction was stopped and absorbance measured at 450 nm. The strength of coloring was proportional to the quantities of human AβpE3-40.

An MSD assay was used to measure AβpE3-42. An MSD standard plate was coated over night at 4° C. with Pyr7.1 as a capture antibody (3 μg/ml per well). Free binding sites were blocked by incubation with 1% blocker A solution prior to incubation for 2 h (900 rpm shaking) with diluted standard (AβpE3-42, 7.1-1000 μg/ml) and test samples. An anti-Aβ42 rabbit polyclonal antibody (produced in-house) was added to the plate (1.5 μg/ml per well) and allowed to incubate for 1 h, followed by a final 1 h incubation with a goat anti-rabbit MDS SULFO-TAG antibody (diluted 1:1000). The plate was washed between blocking and each antibody incubation step. The plate was read in an MSD sector imager where a light signal was generated and measured. The signal strength was correlated to the amount of AβpE3-42 in the sample.

Target binding in human Alzheimer's disease brain by immunohistochemistry: Immunohistochemistry (IHC) analyses were performed on brain tissue from AD and non-demented control. Postmortem human brain tissue of temporal cortex was obtained from the Netherlands Brain Bank (NBB) and had been collected at autopsy with local ethical committee approval.

The mouse anti-human Aβ antibodies 6E10 (Covance #SIG-39320) and 4G8 (Covance #SIG-39200) were used for detection of Aβ pathology in brain sections. As reference antibody, a purified mouse monoclonal anti-AβpE3 IgG1 antibody (Glu3) was used (Biolegend, #822301). Human target binding in AD brain was evaluated for antibodies Pyr7.1 and Pyr12.2, obtained as described in the Examples above.

For IHC staining of Aβ, an automated staining robot and a HRP—3,3′-diaminobenzidine (DAB) based detection system was used (Discovery XT and OmniMap DAB kit, Ventana Medical Systems). IHC analyses were performed on formalin-fixed paraffin embedded tissue sections and on a subset of fresh frozen tissue sections. All tissues were sectioned to 4-8 μm thick sections and mounted onto Superfrost Plus slides (Thermo Fisher). For fresh frozen brain samples, the tissue was sectioned on Superfrost Plus slides and airdried for 30 min, transferred directly to ice cold acetone 50% for 30 s followed by acetone 100% for 5 min and finally 1×PBS for 5 min before being wet-loaded in the Ventana robotic platform. The working concentration used for Pyr7.1 and Pyr12.2 was 1 μg/ml, and the reference antibody Glu3 was used at 0.5 μg/ml. Visualization of the primary/secondary antibody complex was done by addition of hydrogen peroxide and DAB, resulting in an insoluble brown staining precipitate at the site of antibody binding. Counterstaining was done with hematoxylin (HTX). The stained slides were scanned in bright field using a Pannoramic 250 FLASH II slide scanner. The resulting image files were uploaded into a viewer software (Pannoramic Viewer) and adjusted for optimal brightness and contrast for manual assessment of the staining result.

### Results

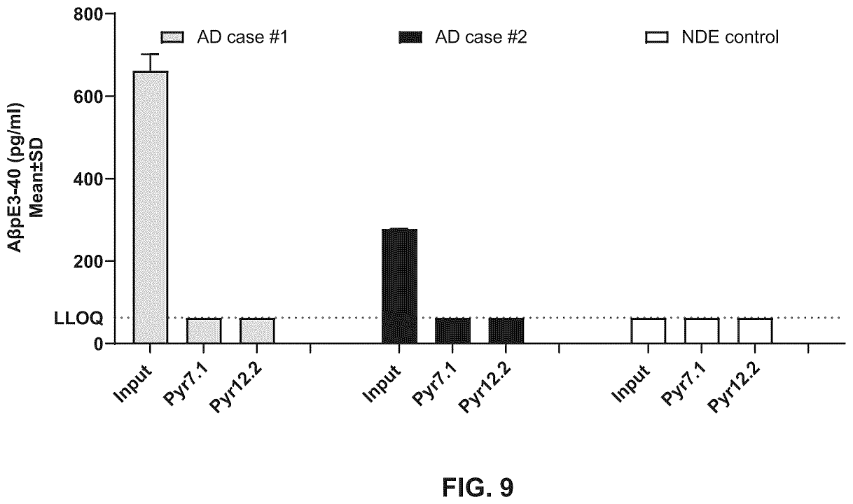

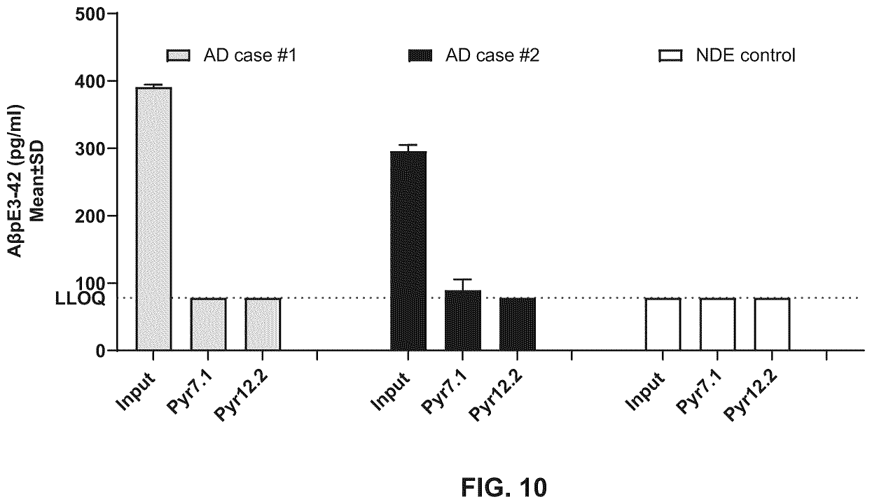

Target binding in human Alzheimer's disease brain extracts by immunoprecipitation: The recombinant antibodies Pyr7.1 and Pyr12.2 were tested for their ability to bind selectively to AβpE3-40 and AβpE3-42 in solution, in human brain extracts from AD patients. Immunoprecipitation (IP) of TBS brain extracts from AD patients using the recombinant antibodies demonstrated depletion of AβpE3-40 (FIG. 9) and AβpE3-42 (FIG. 10) levels by both antibodies. No measurable levels of AβpE3-40 and AβpE3-42 could be detected in brain TBS extract from the NDE control case.

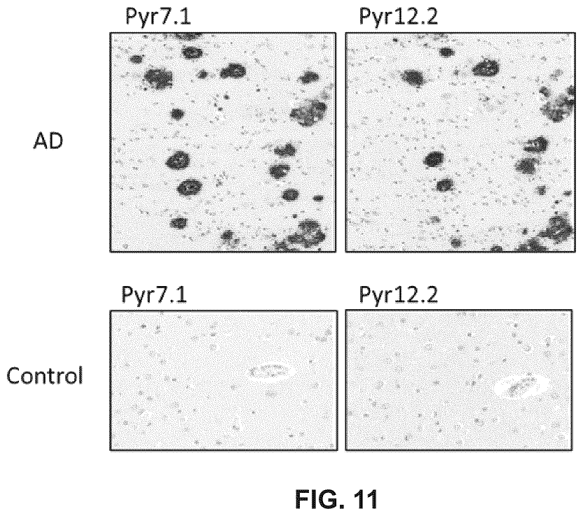

Target binding in human Alzheimer's disease brain by immunohistochemistry: Immunohistochemical staining of brain sections from AD individuals (confirmed to have Aβ pathology by IHC staining with 6E10/4G8, not shown) with recombinant antibodies Pyr7.1 and Pyr12.2 resulted in specific binding by both antibodies to core and diffuse plaques in AD brain, with an identical staining pattern. No binding was observed to NDE control brain. Representative images from immunostaining with Pyr7.1 and Pyr12.2 on adjacent sections from formalin-fixed paraffin embedded AD or NDE control brain are shown in FIG. 11.

### Example 5

### Humanization of Pyr12.2

This example describes the humanization of the murine AβpE3-specific antibody Pyr12.2 described in Examples 2-4, and subsequent production of humanized Pyr12.2 variants.

### Materials and Methods

Humanization: Pyr12.2 was humanized by grafting the CDRs into the IGHV1-46*01 and IKKV2-30*02 human variable domains and making different back-mutations to the mouse residues at various positions. Additional beneficial mutations were performed in the framework regions for one of the variants.

Expression of each variant from transient transfection: The humanized antibodies were expressed in CHO cells and purified by affinity chromatography followed by buffer exchange into phosphate buffered saline (PBS) solution. The purified antibodies were characterized using SDS-PAGE, SEC and UV protein determination.

### Results

Humanization and generation of antibodies: The sequences for humanized VH and VL and the common, human constant regions of the heavy and light chains are given in Table 6 for the two humanized Pyr12.2 variants designated H2L7 and H9L8.

TABLE 6
Amino acid sequences of humanized variants of Pyr12.2

| | | | SEQ |
|---|---|---|---|
| | | | ID |
| Antibody | Region | Amino acid sequence | NO: |
| H2L7 | Heavy chain | | |
| | VH (“VH2”) | EVQLVQSGAEVKKPGASVRLSCKASG | 22 |
| | | YSFTGFTMNWVRQALGQGLEWMGLI | |
| | | NPYNGVTTYNQKFKGRLTMTRDMSTR | |
| | | TVYMDLSSLRYEDTAVYYCTREGNWE | |
| | | GVYWGQGTLVTVSS | |
| | VH-CDR1 | GFTMN | 7 |
| | VH-CDR2 | LINPYNGVTTYNQKFKG | 8 |
| | VH-CDR3 | EGNWEGVY | 3 |
| | Light chain | | |
| | VL (“VL7”) | EIVLTQSPLSLSVTLGQSASISCRSSQS | 23 |
| | | LLDSNGKTYLHWFILRPGQSPRRLIYL | |
| | | VSILDSGVPDRFSGSGSGTDFTLKISR | |
| | | VEAEDVGVYYCVQGTHFPFTFGSGTK | |
| | | LEIK | |
| | VL-CDR1 | RSSQSLLDSNGKTYLH | 10 |
| | VL-CDR2 | LVSILDS | 9 |
| | VL-CDR3 | VQGTHFPFT | 6 |
| H9L8 | Heavy chain | | |
| | VH (“VH9”) | QVQLVQSGPEVKKPGSSVKVSCKASG | 21 |
| | | YSFTGFTMNWVRQTPGKGLEWIGLIN | |
| | | PYNGVTTYNQKFKGRVTITADESTRTA | |
| | | YMELLSLTYEDTAVYYCTREGNWEGV | |
| | | YWGQGTPVTVSA | |
| | VH-CDR1 | GFTMN | 7 |
| | VH-CDR2 | LINPYNGVTTYNQKFKG | 8 |
| | VH-CDR3 | EGNWEGVY | 3 |
| | Light chain | | |
| | VL (“VL8”) | EVVLTQSPLSISVTLGQSASISCRSSQ | 24 |
| | | SLLDSNGKTYLHWFILRPGQSPRRLIY | |
| | | LVSILDSGIPDRFSGSGSGTDFTLKISR | |
| | | VEAEDVGVYYCVQGTHFPFTFGGGTK | |
| | | LEIK | |
| | VL-CDR1 | RSSQSLLDSNGKTYLH | 10 |
| | VL-CDR2 | LVSILDS | 9 |
| | VL-CDR3 | VQGTHFPFT | 6 |
| Human | Constant | ASTKGPSVFPLAPSSKSTSGGTAALG | 31 |
| IgG1 | heavy chain | CLVKDYFPEPVTVSWNSGALTSGVHT | |
| constant | region | FPAVLQSSGLYSLSSVVTVPSSSLGTQ | |
| region | | TYICNVNHKPSNTKVDKKVEPKSCDKT | |
| | | HTCPPCPAPELLGGPSVFLFPPKPKDT | |
| | | LMISRTPEVTCVVVDVSHEDPEVKFN | |
| | | WYVDGVEVHNAKTKPREEQYNSTYR | |
| | | VVSVLTVLHQDWLNGKEYKCAVSNKA | |
| | | LPAPIEKTISKAKGQPREPQVYTLPPS | |
| | | RDELTKNQVSLTCLVKGFYPSDIAVEW | |
| | | ESNGQPENNYKTTPPVLDSDGSFFLY | |
| | | SKLTVDKSRWQQGNVFSCSVMHEAL | |
| | | HNHYTQKSLSLSPGK | |
| Human | Constant | RTVAAPSVFIFPPSDEQLKSGTASVVC | 32 |
| kappa | light | LLNNFYPREAKVQWKVDNALQSGNS | |
| constant | chain | QESVTEQDSKDSTYSLSSTLTLSKADY | |
| region | region | EKHKVYACEVTHQGLSSPVTKSFNRG | |
| | | EC | |

Expression and purification of the humanized antibody variants were performed as described in Example 2 for the recombinant mouse antibodies.

### Example 6

### Characterization of Affinity, Selectivity and Specificity of Humanized Antibodies

This example describes the characterization of the affinity, selectivity and specificity of the humanized Pyr12.2 variant antibodies H₂L7 and H₉L8 generated and produced in Example 5 by inhibition ELISA and SPR.

### Materials and Methods

Aβ monomer species and Aβ protofibrils: The following Aβ peptides were used for characterization of the binding of the humanized antibodies to different Aβ species: AβpE3-40, Aβ1-40, Aβ1-28 and AβpE11-40, all purchased from Bachem, and Aβ2-28, Aβ3-28, Aβ4-28, Aβ5-28 and AβpE11-28, all custom-made by and purchased from Innovagen. Aβ peptides were dissolved in 10 mM NaOH, 0.005% Tween-20, pH>11 at a concentration of 100 μM. Aliquots were made and stored at −80° C. until analysis. All peptides were verified to be monomeric by size-exclusion chromatography.

Protofibrils were prepared using an AβpE3-42 peptide from Bachem. Briefly, the AβpE3-42 peptide was dissolved in 10 mM NaOH, 0.005% Tween-20, pH>11 at a concentration of 100 μM. Protofibrils were prepared by neutralizing the AβpE3-42 peptide to pH 7.4 by adding 1:1 of a 2×PBS buffer to a final concentration of 50 μM. The peptide was incubated for ~30 min at 37° C. for protofibril formation and purified from remaining monomers by HPLC on a Superdex 75 Increase 3.2/300 size-exclusion column using a mobile-phase of 1×PBS, 0.1% Tween-20, pH 7.4. Prior to injection, the protofibril reaction was centrifuged at 16000×g for 5 min at 4° C. to remove insoluble fibrils. The void peak containing the AβpE3-42 protofibrils was collected and the concentration determined using SEC and a calibration curve of an Aβ protofibril standard with a known concentration. The same procedure was used to produce Aβ1-42 protofibrils from the corresponding Aβ1-42 monomer (Bachem).

Specificity evaluation and IC₅₀ determination by inhibition ELISA: The specificity of humanized antibodies H2L7 and H9L8 towards AβpE3-28 compared to N-terminally intact Aβ (Aβ1-28) and different N-truncated forms of Aβ (Aβ2-28, Aβ3-28, Aβ4-28, Aβ5-28 and AβpE11-28 monomers) was evaluated by inhibition ELISA. The humanized antibodies were incubated at a fixed concentration (0.5 μg/ml) with titrating concentrations of the different Aβ antigens. After incubation for 45 min at 900 rpm to reach equilibrium, the antibody-Aβ samples were added to a blocked and washed ELISA plate with an AβpE3-40 coat (0.5 μM). The samples were incubated on the plate for 25 min without shaking followed by washing, incubation with detection antibody, another washing step and finally incubation with alkaline phosphatase substrate. Optical density at 405 nm was read, and the collected data was analyzed using a four-parameter variable slope non-linear fit to determine IC₅₀ values.

Selectivity evaluation and IC₅₀ determination by inhibition ELISA: The binding of humanized antibodies H2L7 and H9L8 to AβpE3-40 and AβpE3-42 protofibrils and their selectivity towards Aβ1-40 monomers and Aβ1-42 protofibrils were evaluated by inhibition ELISA. The humanized antibodies were incubated at a fixed concentration (0.1 μg/ml) with titrating concentrations of the different Aβ antigens. After incubation for 45 min at 900 rpm to reach equilibrium, the antibody-Aβ samples were added to a blocked and washed ELISA plate with an AβpE3-40 coat (0.5 μM). The samples were incubated on the plate for 25 min without shaking followed by washing, incubation with detection antibody, another washing step and finally incubation with alkaline phosphatase substrate. Optical density at 405 nm was read, and the collected data was analyzed using a four-parameter variable slope non-linear fit to determine IC₅₀ values.

Affinity evaluation and Ko determination by surface plasmon resonance: Binding interactions between antigens and antibodies were evaluated by SPR using a Biacore 8K instrument (Cytiva) according to standard procedures. The binding of humanized antibodies H2L7 and H9L8 to AβpE3-40 monomers and AβpE3-42 protofibrils was evaluated.

Single cycle kinetics with the antibodies immobilized on a CM5 chip was used to measure the binding of the antibodies to the AβpE3-40 monomer. For the measurements, 5 μg/ml of analyte antibody was immobilized on the chip. The monomer was then injected over the chip using a 2-fold dilution in five steps starting at 250 nM, using 2 min injection of every antibody concentration and a 20 min dissociation time. Regeneration of the surface between cycles was done by injecting 30 μl 3 M MgCl₂. The binding data was fitted to a 1:1 interaction model.

Single cycle kinetics was also used to measure binding of the antibodies to AβpE3-42 protofibrils. The AβpE3-42 protofibrils (138 ng/ml) were coupled to a CM5 chip using general Biacore coupling chemistry (immobilization low levels). For binding to AβpE3-42 protofibrils, the antibodies were injected over the chip using a 4-fold dilution series in five steps starting at 150 nM, using 2 min injection of every antibody concentration and a 20 min dissociation time. Regeneration of the surface between cycles was done by injecting 30 μl 10 mM glycine-HCl pH 1.7. The binding data was fitted to a 1:1 interaction model. The use of the 1:1 interaction model gives the Ko for the binding of the antibodies to Aβ monomers and an apparent Ko for the binding of the antibodies to Aβ protofibrils.

In all SPR experiments, 1×HBS-EP+ (Cytiva, cat. no. BR100669) was used to dilute antibodies and target antigens. Experiments were performed at 25° C.

### Results

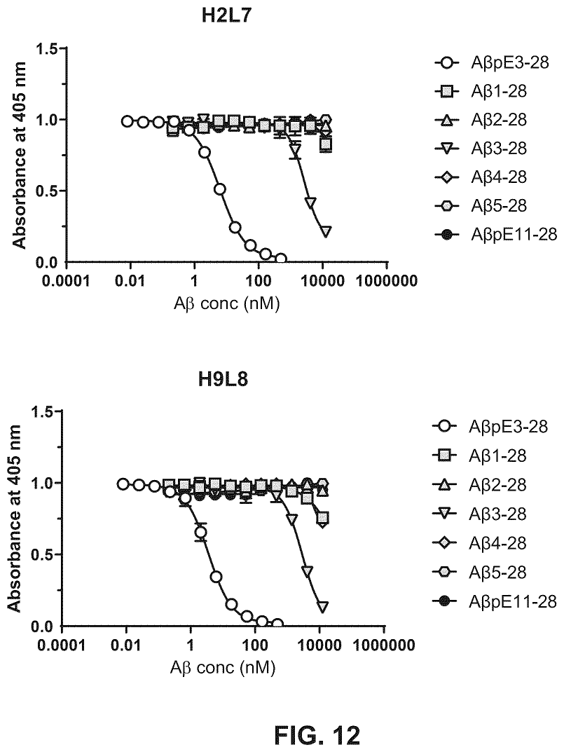

Specificity evaluation and IC₅₀ determination by inhibition ELISA: The specificity of humanized antibodies H2L7 and H9L8 towards AβpE3-28 compared to N-terminally intact Aβ (Aβ1-28) and different N-truncated forms of Aβ (Aβ2-28, Aβ3-28, Aβ4-28, Aβ5-28 and AβpE11-28 monomers) was evaluated by inhibition ELISA. Humanized antibodies H2L7 and H9L8 demonstrated highest binding to AβpE3-28 monomer in solution, with some cross-reactivity to Aβ3-28 (FIG. 12). None of the antibodies bound Aβ1-28, Aβ2-28, Aβ4-28, Aβ5-28 or AβpE11-28 monomers in solution at concentrations up to 12.5 μM, suggesting the IC₅₀ values were >12.5 μM for these antibodies. The calculated IC₅₀ values are listed in Table 7.

TABLE 7
Specificity evaluation using inhibition ELISA (Mean ± SD)

| | Aβ1-28 | Aβ2-28 | Aβ3-28 | AβpE3-28 | Aβ4-28 | Aβ5-28 | AβpE11-28 |
|---|---|---|---|---|---|---|---|
| Ab | IC₅₀ (nM) | IC₅₀ (nM) | IC₅₀ (nM) | IC₅₀ (nM) | IC₅₀ (nM) | IC₅₀ (nM) | IC₅₀ (nM) |
| H2L7 | >12500 | >12500 | 1872 ± 1387 | 5.5 ± 2.8 | >12500 | >12500 | >12500 |
| H9L8 | >12500 | >12500 | 1788 ± 1333 | 3.5 ± 0.8 | >12500 | >12500 | >12500 |

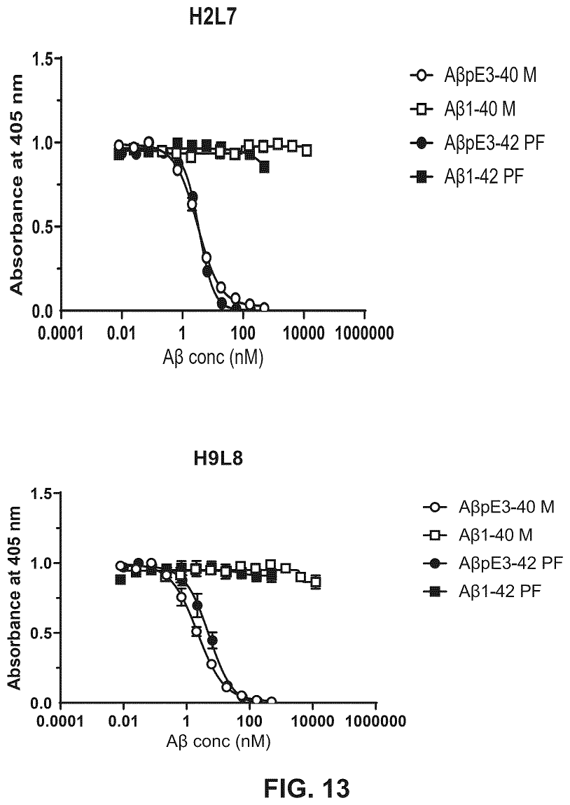

Selectivity evaluation and IC₅₀ determination by inhibition ELISA: The binding of the humanized antibodies H2L7 and H9L8 to AβpE3-40 monomers and AβpE3-42 protofibrils and selectivity versus Aβ1-40 monomers and Aβ1-42 protofibrils was evaluated using inhibition ELISA. Humanized antibodies H2L7 and H9L8 demonstrated binding to AβpE3-40 monomer and AβpE3-42 protofibril in solution (FIG. 13). None of the antibodies bound Aβ1-40 monomer in solution at concentrations up to 12.5 μM, suggesting the IC₅₀ values were >12.5 μM for these antibodies. None of the antibodies bound Aβ1-42 protofibrils in solution at concentrations up to 500 nM, suggesting the IC₅₀ values were >500 nM for these antibodies. The calculated IC₅₀ values are listed in Table 8.

TABLE 8
Selectivity analysis using inhibition ELISA (Mean ± SD)

| | AβpE3-40 | AβpE3-42 | Aβ1-40 | Aβ1-42 |
|---|---|---|---|---|
| | monomer | protofibril | monomer | protofibril |
| | IC₅₀ | IC₅₀ | IC₅₀ | IC₅₀ |
| Antibody | (nM) | (nM) | (nM) | (nM) |
| H2L7 | 2.6 ± 0.7 | 2.5 ± 0.9 | >12500 | >500 |
| H9L8 | 2.1 ± 0.3 | 4.9 ± 1.1 | >12500 | >500 |

Affinity evaluation and Ko determination by surface plasmon resonance: The binding of humanized antibodies H2L7 and H9L8 to AβpE3-monomers and AβpE3-42 protofibrils was evaluated in SPR and their K_{D} values were determined.

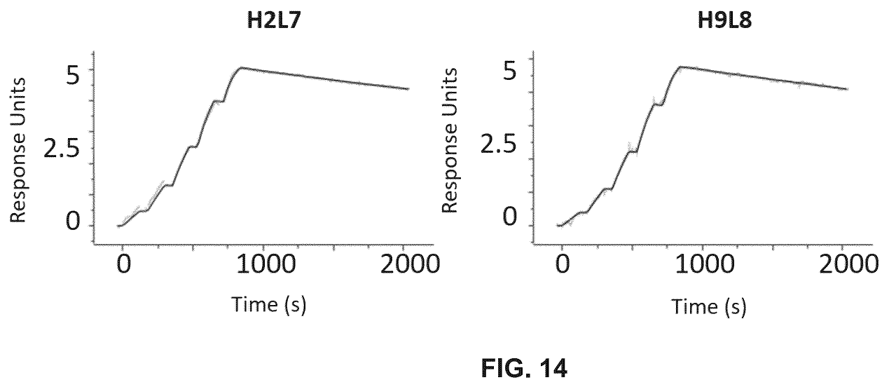

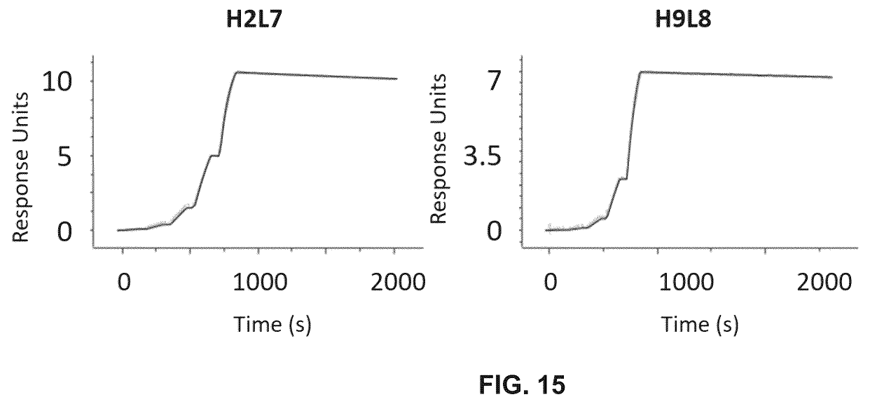

Both antibodies demonstrated binding to AβpE3-40 monomers and AβpE3-42 protofibrils. The calculated kₐ, k_{d} and (apparent) K_{D} values are shown in Table 9 and Table 10 below. Representative sensorgrams are shown in FIG. 14 and FIG. 15.

TABLE 9
SPR analysis of binding to AβpE3-40 monomers

| | AβpE3-40 monomer | | |
|---|---|---|---|
| | kₐ (M⁻¹s⁻¹) | Kₐ (s⁻¹) | K_{D} (nM) |
| Antibody | Mean ± SD | Mean ± SD | Mean ± SD |
| H2L7 | 5.05 ± 0.81 e4 | 1.25 ± 0.24 e−4 | 2.54 ± 0.60 |
| H9L8 | 5.37 ± 1.69 e4 | 1.39 ± 0.44 e−4 | 2.70 ± 0.67 |

TABLE 10
SPR analysis of binding to AβpE3-42 protofibrils

| | AβpE3-42 protofibril | | |
|---|---|---|---|
| | | | Apparent |
| | kₐ (M⁻¹s⁻¹) | Kₐ (s⁻¹) | K_{D} (nM) |
| Antibody | Mean ± SD | Mean ± SD | Mean ± SD |
| H2L7 | 1.07 ± 0.08 e5 | 1.79 ± 0.84 e−5 | 173 ± 99.4 |
| H9L8 | 4.43 ± 0.34 e4 | 2.05 ± 0.51 e−5 | 464 ± 117 |

### Example 7

Target Binding of Humanized Antibodies in Brain from Human Alzheimer's Disease Patients and Non-Demented Controls

This example describes target binding of humanized antibodies H2L7 and H9L8, generated as described in Example 5 and tested by immunoprecipitation on human brain extracts and immunohistochemistry on human brain sections from AD patients and NDE controls.

### Materials and Methods

Target binding in human Alzheimer's disease brain extracts by immunoprecipitation: Antibody binding to target in human AD brain was analyzed by immunoprecipitation, which is a method for removal of target protein in a sample using an antibody specific for the target molecule. Briefly, each of the humanized antibodies was incubated with magnetic Protein A Dynabeads and soluble 16000×g TBS brain extracts from an AD case. The TBS brain extracts were prepared as described in Example 4. The bead-bound target was separated by a magnet and eluted from the beads using 70% formic acid. After neutralization, the pellet (IP fraction) was analyzed using in house developed MSD assay for measurements of total AβpE3-x levels. Briefly, MSD GOLD 96-well small spot streptavidin plate was coated with biotinylated Pyr12.2 antibody for 1 h at RT. After a washing step, free binding sites were blocked by incubation with Diluent 35. The plate was washed again and incubated further for 2 h (900 rpm shaking) with a dilution series of a standard (AβpE3-40 monomer, 3.125-400 μg/ml) and with test samples. After another washing step, detection antibody anti-Aβ 4G8 SULFO tag conjugated was added to the plate for 1 h at a concentration of 1 μg/ml. Finally, the plate was washed and read using an MSD sector imager (S 600 MM, MSD). Obtained signal was correlated to the amount of AβpE3-x in the sample.

Target binding in human Alzheimer's disease brain by immunohistochemistry: Immunohistochemistry (IHC) analyses were performed on brain tissue from AD patients. Postmortem human brain tissue of temporal cortex was obtained from the Netherlands Brain Bank (NBB) and had been collected at autopsy with local ethical committee approval.

The mouse anti-human Aβ antibodies 6E10 (Covance #SIG-39320) and 4G8 (Covance #SIG-39200) were used for detection of Aβ pathology in brain sections. Human target binding in AD brain was evaluated for humanized antibodies H2L7 and H9L8.

For IHC staining of Aβ, an automated staining robot and a HRP—3,3′-diaminobenzidine (DAB) based detection system was used (Discovery XT and OmniMap DAB kit, Ventana Medical Systems). IHC analyses were performed on fresh frozen tissue sections. All tissues were sectioned in 4-8 μm sections which were mounted onto Superfrost Plus slides (ThermoFisher). The sections were airdried for 30 min, transferred directly to ice cold acetone 50% for 30 s followed by acetone 100% for 5 min and finally 1×PBS for 5 min before being wet-loaded in the Ventana robotic platform. The working concentration used for tested antibodies H2L7 and H9L8 and for reference antibodies 6E10 and 4G8 was 1 μg/ml. Visualization of the primary/secondary antibody complex was done by addition of hydrogen peroxide and DAB, resulting in an insoluble brown staining precipitate at the site of antibody binding. Counterstaining was done with hematoxylin (HTX). The stained slides were scanned in bright field using a Pannoramic 250 FLASH II slide scanner. The resulting image files were uploaded into a viewer software (Pannoramic Viewer) and adjusted for optimal brightness and contrast for manual assessment of the staining result.

### Results

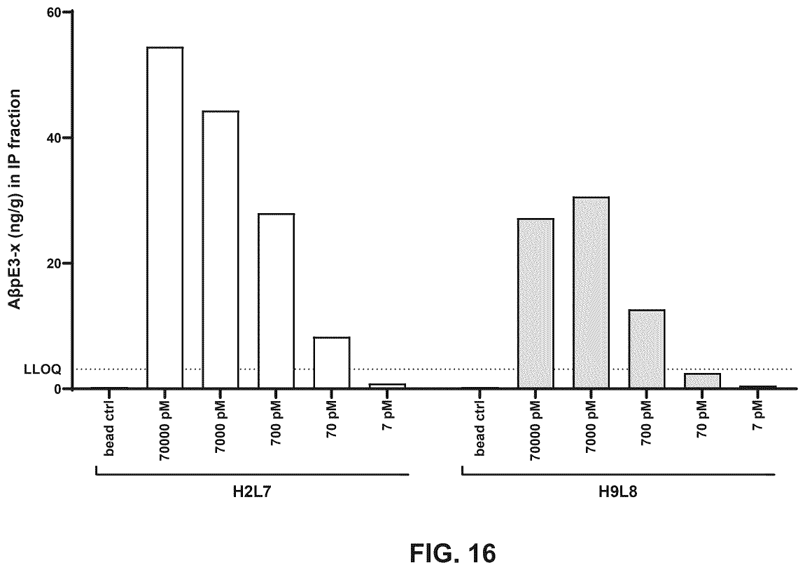

Target binding in human Alzheimer's disease brain extracts by immunoprecipitation: The humanized antibodies H2L7 and H9L8 were tested for their ability to bind to AβpE3 in solution, in brain extracts from human AD patients. Immunoprecipitation (IP) of TBS brain extracts from AD patients using the humanized antibodies demonstrated a concentration-dependent IP of AβpE3-x by both antibodies (FIG. 16).

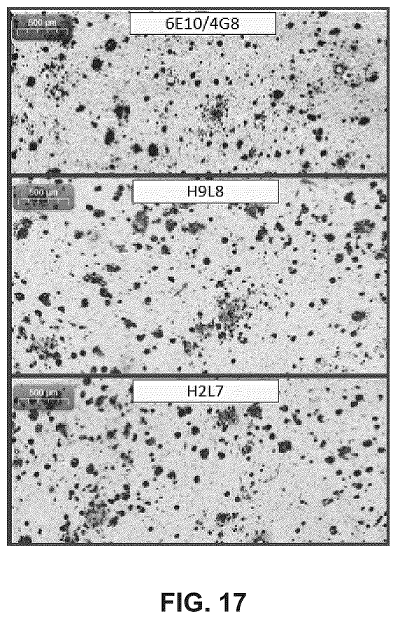

Target binding in human Alzheimer's disease brain by immunohistochemistry: Immunohistochemical staining of brain sections from AD individuals (confirmed to have Aβ pathology by IHC staining with 6E10/4G8) with humanized antibodies H2L7 and H9L8 resulted in specific binding by both antibodies to core and diffuse plaques in AD brain, with an identical staining pattern. No binding was observed to NDE control brain (data not shown). Representative images from immunostaining with H2L7 and H9L8 on adjacent sections from fresh-frozen AD brain are shown in FIG. 17.

### Example 8

### Characterization of Functional Effects of Humanized Antibodies

This example describes the functional effects of humanized AβpE3 antibodies H2L7 and H9L8 generated and produced in Example 5. The ability of the humanized antibodies to inhibit aggregation of AβpE3 and to clear amyloid plaques ex vivo in AD brain sections was evaluated.

### Materials and Methods

Inhibition of aggregation of AβpE3-42: The effect of the humanized antibodies on aggregation of AβpE3-42 monomers was evaluated in an aggregation assay using thioflavin T (ThT, Sigma T3516). AβpE3-42-NH4+ monomers (Bachem, H4916, 2 μM) were mixed with ThT (5 μM) and with either H2L7 or H9L8 humanized antibody (25-800 nM) or an IgG1 isotype control antibody (CrownVivo, C-00012, 800 nM) in phosphate buffer saline (PBS) pH 8.2, 200 mM NaCl, 10 μM EDTA in 384-well plates (Thermo scientific #242764) on ice. The plates were then transferred to a Tecan SPARK enhanced plate reader equipped with a 448±7 nm excitation filter and a 485±20 nm emission filter at 37° C. for recordings of ThT fluorescence over 36-72 hrs. The data was background corrected and the maximum fluorescence plotted against antibody concentration to calculate the IC₅₀. The experiment was repeated 3-5 times. Statistical significance was tested with two-way ANOVA.

Ex vivo phagocytosis in AD brain: An ex vivo phagocytosis assay was used to investigate whether the humanized antibodies could induce plaque clearance by macrophages. Fresh frozen AD brain tissue was cryosectioned (20 μm) and sections were collected onto poly-D-lysine (Gibco A38904-01, 50 μg/ml) coated 12 mm glass coverslips. Sections were then incubated with humanized antibody (1 μg/ml) or an IgG1 isotype control antibody (CrownVivo, C-00012, 1 μg/ml) for 1 h at 37° C. 5% CO₂. Sections were then washed once and incubated for 24 h with 5×10⁵ to 1×10⁶ primary human macrophages isolated from buffy coats. Plaque clearance was evaluated after immunohistochemistry with the mouse anti-human Aβ antibodies 6E10 (Covance #SIG-39320) and 4G8 (Covance #SIG-39200) by measuring the immunopositive area on each section. Experiments were repeated 2 to 5 times with macrophages isolated from different buffy coats. Statistical significance was tested with one-way ANOVA.

### Results

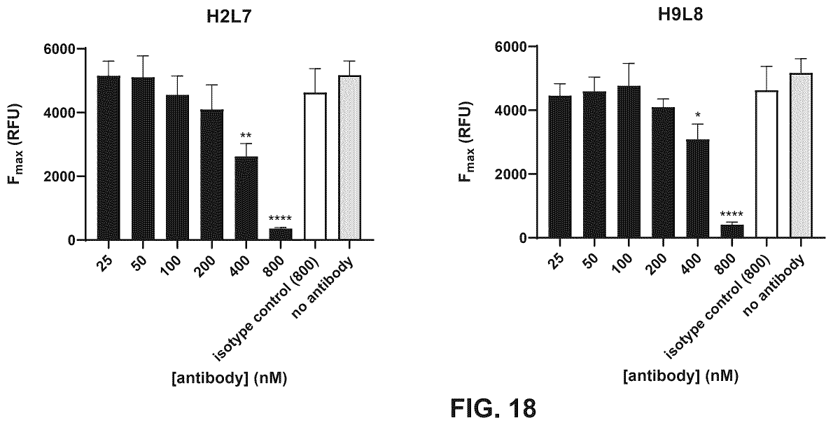

Inhibition of aggregation of AβpE3-42: The ability of the humanized antibodies H2L7 and H9L8 to inhibit aggregation of AβpE3-42 was evaluated. Both H2L7 and H9L8 concentration-dependently inhibited fibril formation of AβpE3-42, as demonstrated by the decrease in the maximum ThT fluorescence signal (Fmax) in the presence of the antibodies (FIG. 18).

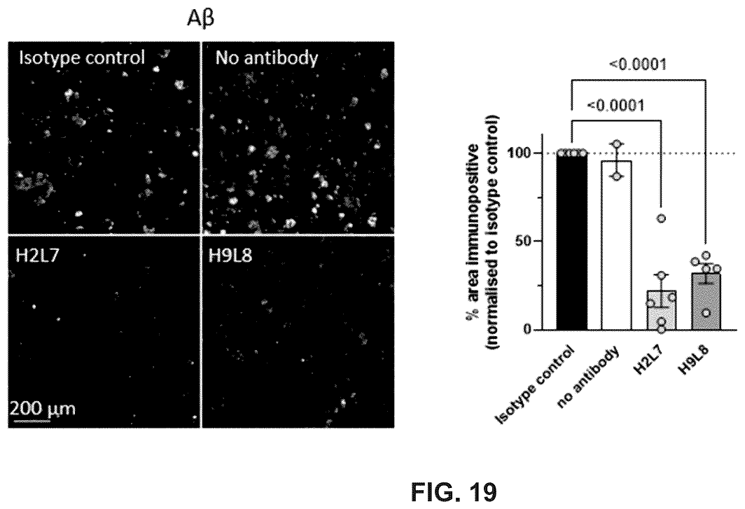

Ex vivo phagocytosis in AD brain: The ability of the humanized antibodies H2L7 and H9L8 to induce clearance of Aβ plaques by macrophages in AD brain was evaluated. Compared to negative control samples pre-incubated without antibodies or with an isotype control IgG1 antibody, Aβ plaques were significantly reduced after pre-incubation with H2L7 or H9L8. The results indicate that both antibodies can induce plaque clearance by macrophages (FIG. 19).

### Example 9

### Pharmacokinetic Profile of Murine and Humanized AβpE3 Antibodies

This example describes the pharmacokinetic (PK) profile in mouse after dosing with murine antibody Pyr12.2, generated and produced as described in Example 2, or with humanized antibodies H2L7 and H9L8, generated and produced as described in Example 5.

### Materials and Methods

Administration of antibody and sample collection: Each respective antibody was administered intravenously (i.v.) at a dose of 10 mg/kg via the tail vein to 8 weeks old female C57BL/6J mice (five mice per antibody). Blood samples were collected at 5 min, 4 h, 24 h, 72 h, 168 h (7 days), 336 h (14 days), 672 h (28 days) and 840 h (35 days) after i.v. injection. Blood was collected into Microvette EDTA tubes and put on wet ice immediately after collection. The samples were centrifuged at 2400×g for 10 min at +4° C. shortly after collection (within 30 min). Plasma was collected and stored at −80° C. until bioanalysis.

Determination of antibody concentrations in plasma: Concentrations of Pyr12.2 were determined in EDTA plasma samples using an MSD based method. Briefly, MSD standard 96-well plates were coated with 0.5 μM monomeric AβpE3-40 (Bachem) overnight at 4° C. Free binding sites were blocked by incubation with 1% Blocker A (MSD Blocker A in 1×PBS-Tween 20) for 1 h at RT with shaking. Washing was performed before blocking and before each subsequent incubation step. Standard and plasma samples were added and incubated for 2 h at RT with shaking. Bound antibodies were detected by incubation with an MSD SULFO-TAG labelled goat anti-mouse IgG antibody (R32AC-1, 0.5 μg/ml) for 1 h at RT with shaking. Read buffer T (MSD 2×) was added and the plates were read using an MSD sector imager. The signal strength was correlated to the amount of Pyr12.2 in the samples.

Concentration levels of H2L7 and H9L8 were determined in EDTA plasma samples using a commercial kit from MSD for measuring Human/NHP IgG (cat no: K150JLD). Standard and plasma samples were added to a precoated anti-human-IgG MSD plate and incubated for 2 h in RT with shaking. An anti-human/NHP IgG antibody conjugated with a SULFO-TAG label was added after a washing step and incubated at 2 h, after which the MSD read buffer was added after a washing step. When read in an MSD sector imager a light signal was generated and measured. The signal strength was correlated to the amount of the H2L7 or H9L8 in the samples.

Pharmacokinetic analysis: The software Phoenix WinNonlin was used for non-compartment analysis (NCA) of the plasma concentration data. Individual observed plasma concentration versus time profiles were subjected to PK evaluation. Nominal dose and time points were used in the analysis. The maximum concentration, Cmax, was directly derived from the observed concentrations versus time curves. The calculation method in the NCA was set to Linear up log down, where linear trapezoidal rule is used when concentration versus time data is increasing, and the logarithmic trapezoidal rule is used when the concentration data is decreasing. Calculated parameters included the area under the concentration versus time curve (AUC) to the last observed time point (AUClast) or to infinity (AUCinf), calculated as AUClast+Ct/λz, where Ct is the observation at the last time point and λz is the elimination rate constant estimated using log-linear regression during the terminal elimination phase. Terminal half-life (t½) was calculated as ln(2)/λz, clearance (CL) was calculated as Dose/AUCinf.

### Results

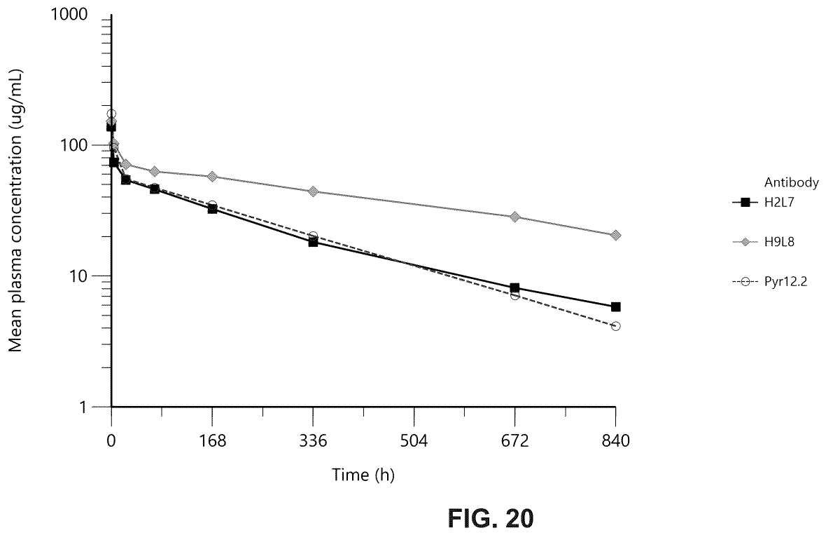

The plasma PK profiles for the murine Pyr12.2 antibody and humanized antibodies H2L7 and H9L8 were evaluated after a single i.v. bolus injection into C57BL/6 mice. The profiles of plasma concentration as a function of time are shown in FIG. 20 and the calculated PK parameters are shown in Table 11. While H2L7 showed a similar plasma PK profile in mouse as the murine antibody Pyr12.2, the H9L8 variant surprisingly demonstrated a better plasma PK profile with a longer half-life, a higher total exposure (AUC) and lower clearance (CL).

TABLE 11
Summary of PK parameters (Mean)

| | Half-life | Cmax | AUCinf | CL |
|---|---|---|---|---|
| Antibody | (days) | (μg/ml) | (mg/ml*h) | (ml/h/kg) |
| Pyr12.2 | 9.3 | 173 | 19.3 | 0.52 |
| H2L7 | 11.5 | 140 | 19.6 | 0.54 |
| H9L8 | 19.6 | 152 | 49.7 | 0.20 |

### Example 10

Generation of Humanized Antibodies with an Improved Pharmacokinetic Profile

This example describes the generation of variants of humanized antibody H2L7 having an improved pharmacokinetic (PK) profile.

### Material and Methods

Design and expression of H2L7 variants: The HC and LC sequences of the humanized antibodies H2L7 and H9L8 of the preceding Examples were compared in order to identify positively charged amino acids that could potentially cause the difference in PK profile between the two antibodies (Table 11). Sequences were also compared to the murine parental antibody Pyr12.2. The structures for H2L7 and H9L8 were modeled in silico, and surface analysis was performed. Amino acids in H2L7 that were identified to contribute to positively charged patches were mutated to neutral amino acids. Different mutations were combined to generate several new antibodies. New surface analyses were performed for mutated antibodies. A total of six antibodies with mutations that reduced positively charged patches in the surface analysis were expressed and purified as described in Example 5 for H2L7 and H9L8, with an additional preparative grade SEC step.

Administration of antibody and sample collection: The antibody variants were administered at a 10 mg/kg dose i.v. via the tail vein to 8 weeks old female C57BL/6J mice (five mice per antibody). Blood samples were collected at 5 min, 4 h, 24 h, 72 h, 168 h (7 days), 336 h (14 days), 672 h (28 days) and 840 h (35 days) after the i.v. injection. Blood was collected into Microvette EDTA tubes and put on wet ice immediately after collection. The samples were centrifuged at 2400×g for 10 minutes at 4° C. shortly after collection (within 30 min). Plasma was collected and stored at −80° C. until bioanalysis.

Determination of antibody concentration in plasma: Concentrations of the antibody variants were determined in EDTA plasma samples using a commercial kit from MSD for measuring Human/NHP IgG (catalog number: K150JLD), as described in Example 9 for H2L7 and H9L8.

Pharmacokinetic analysis: Individual, observed profiles of plasma concentration versus time were subjected to PK evaluation as described in Example 9 for H2L7 and H9L8.

### Results

Design of H2L7 variants with an improved pharmacokinetic profile: From the sequence alignment of VH2 (SEQ ID NO:22) and VH9 (SEQ ID NO:21), residues R71 and R83 found in VH2 were considered to be highly likely to contribute to the higher clearance for H2L7 in comparison to VH9 in H9L8 (comprising A71 and T83). K12 in VH2 was also considered as a candidate for mutation, because the corresponding residue at this position in the parental murine Pyr12.2 antibody is a neutral valine residue. It is worth noting that VH9 also has a K12 residue, and thus the discrepancy in clearance between H2L7 and H9L8 may not stem from this position. With respect to the light chain, there were no obvious residue differences between VL7 (SEQ ID NO:23) and VL8 (SEQ ID NO:24) that were deemed to contribute to the increased net positive charge in H2L7 compared to H9L8. Based on a sequence alignment, it was concluded that the VH domain was likely responsible for the notable difference in clearance between H2L7 and H9L8. It was therefore hypothesized that making mutations in VH2 to remove the positively charged residues could reduce clearance of H2L7 to be more in line with the clearance observed for H9L8.

Protein surface analysis of H2L7 and H9L8 revealed distinct, positively charged regions in H2L7 which are either missing or significantly reduced in H9L8. R83 in the VH domain of H2L7 contributes to a large positively charged patch which is absent in H9L8, because the corresponding residue at that position (threonine, T83) is a neutral amino acid. Similarly, R71 in VH of H2L7 contributes to a positively charged patch, while the corresponding residue in H9L8 is alanine (A71) which does not contribute to a positive protein surface patch. Also, position 12 in the VH domain of H2L7 was mutated from the K12 present in both VH2 and VH9 back to the neutral V12 present in the murine ancestor Pyr12.2. This could also contribute to a reduced overall positive protein surface.

Taking the above analysis into account, variants of H2L7 with the following mutations in the heavy chain variable domain (VH) were postulated to have an improved PK: H2L7-R71A, H2L7-R83T, H2L7-K12V/R71A, H2L7-K12V/R83T, H2L7-R71A/R83T and H2L7-K12V/R71A/R83T. The VH sequences are shown in Table 12. The amino acid sequences of the VL and constant domains of the variant antibodies were identical to the sequences of the corresponding domains in H2L7 given in Table 6. Of these, H2L7-R71A, H2L7-R83T, H2L7-K12V/R71A, H2L7-K12V/R83T, H2L7-R71A/R83T and H2L7-K12V/R71A/R83T were produced.

TABLE 12
Amino acid sequences
of VH domain variants of H2L7

| | | SEQ |
|---|---|---|
| | | ID |
| Antibody | VH domain amino acid sequence | NO: |
| H2L7-R71A | EVQLVQSGAEVKKPGASVRLSCKASGYSFTG | 16 |
| | FTMNWVRQALGQGLEWMGLINPYNGVTTYN | |
| | QKFKGRLTMTADMSTRTVYMDLSSLRYEDTA | |
| | VYYCTREGNWEGVYWGQGTLVTVSS | |
| H2L7-R83T | EVQLVQSGAEVKKPGASVRLSCKASGYSFTG | 17 |
| | FTMNWVRQALGQGLEWMGLINPYNGVTTYN | |
| | QKFKGRLTMTRDMSTRTVYMDLSSLTYEDTA | |
| | VYYCTREGNWEGVYWGQGTLVTVSS | |
| H2L7-K12V/ | EVQLVQSGAEVVKPGASVRLSCKASGYSFTG | 15 |
| R71A | FTMNWVRQALGQGLEWMGLINPYNGVTTYN | |
| | QKFKGRLTMTADMSTRTVYMDLSSLRYEDTA | |
| | VYYCTREGNWEGVYWGQGTLVTVSS | |
| H2L7-K12V/ | EVQLVQSGAEVVKPGASVRLSCKASGYSFTG | 18 |
| R83T | FTMNWVRQALGQGLEWMGLINPYNGVTTYN | |
| | QKFKGRLTMTRDMSTRTVYMDLSSLTYEDTA | |
| | VYYCTREGNWEGVYWGQGTLVTVSS | |
| H2L7-R71A/ | EVQLVQSGAEVKKPGASVRLSCKASGYSFTG | 20 |
| R83T | FTMNWVRQALGQGLEWMGLINPYNGVTTYN | |
| | QKFKGRLTMTADMSTRTVYMDLSSLTYEDTA | |
| | VYYCTREGNWEGVYWGQGTLVTVSS | |
| H2L7-K12V/ | EVQLVQSGAEVVKPGASVRLSCKASGYSFTG | 19 |
| R71A/R83T | FTMNWVRQALGQGLEWMGLINPYNGVTTYN | |
| | QKFKGRLTMTADMSTRTVYMDLSSLTYEDTA | |
| | VYYCTREGNWEGVYWGQGTLVTVSS | |

### Evaluation of Pharmacokinetic Profile of H2L7 and Variants in Mouse:

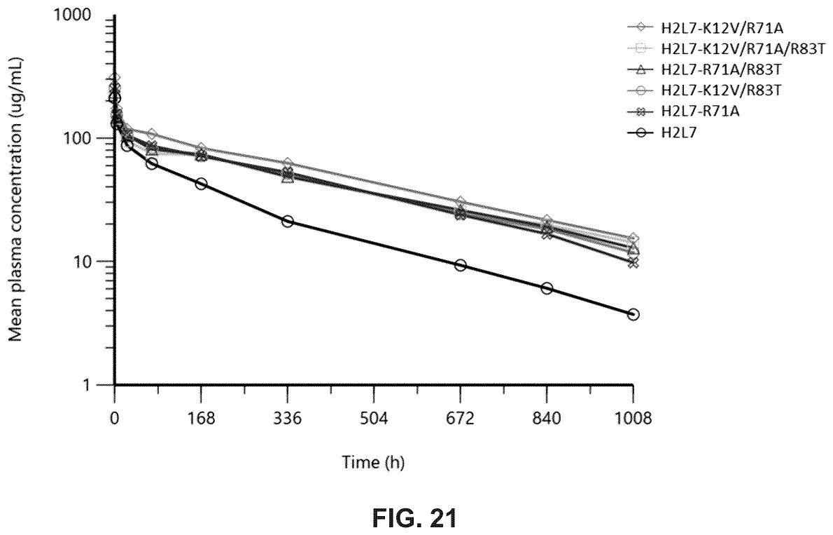

The plasma PK profile for H2L7 and five H2L7 variants after a single i.v. bolus injection into C57BL/6 mice was evaluated. The profiles of plasma concentration versus time are shown in FIG. 21 and the calculated PK parameters are shown in Table 13. All variants of H2L7 showed an improved plasma PK profile in mouse, exhibiting longer half-life, higher total exposure (AUC) and lower clearance (CL) than the original H2L7 antibody.

TABLE 13
Summary of PK parameters (Mean)

| | Half-life | AUCinf | Cmax | CL |
|---|---|---|---|---|
| Antibody | (days) | (mg/ml*h) | (μg/ml) | (ml/h/kg) |
| H2L7 | 10.8 | 24.7 | 211.0 | 0.41 |
| H2L7-K12V/R71A | 14.0 | 60.4 | 310.1 | 0.17 |
| H2L7-R71A | 12.5 | 47.9 | 251.1 | 0.21 |
| H2L7-K12V/R83T | 13.4 | 49.2 | 256.8 | 0.21 |
| H2L7-R71A/R83T | 14.3 | 50.2 | 234.2 | 0.20 |
| H2L7-K12V/R71A/R83T | 15.2 | 50.4 | 216.9 | 0.20 |

### Example 11

### Affinity, Selectivity and Specificity of H2L7 Variant Antibodies

This example describes characterization, by inhibition ELISA and SPR, of the affinity, selectivity and specificity of the H2L7 variant antibodies generated and produced as described in Example 10.

### Materials and Methods

Aβ monomer species and Aβ protofibrils: The Aβ monomer species as described in Example 6 were used for characterization of binding to monomeric Aβ species. AβpE3-42 and Aβ1-42 protofibrils were prepared as described in Example 6 and used for characterization of binding to aggregated Aβ species.

Specificity evaluation and IC₅₀ determination by inhibition ELISA: The specificity towards AβpE3-28 compared to N-terminally intact Aβ (Aβ1-28) and different N-truncated forms of Aβ (Aβ2-28, Aβ3-28, Aβ4-28, Aβ5-28 and AβpE11-28 monomers) was evaluated by inhibition ELISA as described for H2L7 and H9L8 in Example 6.

Selectivity evaluation and IC₅₀ determination by inhibition ELISA: The binding of the antibodies to AβpE3-40 and AβpE3-42 protofibrils and selectivity against Aβ1-40 monomers and Aβ1-42 protofibrils was evaluated by inhibition ELISA as described for H2L7 and H9L8 in Example 6.

Affinity evaluation and Ko determination by surface plasmon resonance: Binding interactions between antigens and antibodies were evaluated by SPR using a Biacore 8K instrument (Cytiva) according to standard procedures. The binding of the H2L7 variant antibodies to AβpE3-40 monomers and AβpE3-42 protofibrils was evaluated as described for H2L7 and H9L8 in Example 6, with the exception of using immobilization of 10 μg/ml of analyte antibody on the chip for measurement of binding to AβpE3-40 monomer.

### Results

Specificity evaluation and IC₅₀ determination by inhibition ELISA: The specificity of H2L7, H2L7-K12V/R71A and H2L7-K12V/R83T towards AβpE3-28 compared to N-terminally intact Aβ (Aβ1-28) and different N-truncated forms of Aβ (Aβ2-28, Aβ3-28, Aβ4-28, Aβ5-28, and AβpE11-28 monomers) was evaluated by inhibition ELISA. H2L7 and both H2L7 variants demonstrated the highest binding to AβpE3-28 monomer in solution, with some cross-reactivity to Aβ3-28. The calculated IC₅₀ values are listed in Table 14.

TABLE 14
Specificity evaluation using inhibition ELISA

| | Aβ1-28 | Aβ2-28 | Aβ3-28 | AβpE3-28 | Aβ4-28 | Aβ5-28 | AβpE11-28 |
|---|---|---|---|---|---|---|---|
| Antibody | IC₅₀ (nM) | IC₅₀ (nM) | IC₅₀ (nM) | IC₅₀ (nM) | IC₅₀ (nM) | IC₅₀ (nM) | IC₅₀ (nM) |
| H2L7 | >12500 | >12500 | 2347 | 3.6 | >12500 | >12500 | >12500 |
| H2L7-K12V/R71A | 10179 | >12500 | 2820 | 3.0 | >12500 | >12500 | >12500 |
| H2L7-K12V/R83T | >12500 | >12500 | 2260 | 5.0 | >12500 | >12500 | >12500 |

Selectivity evaluation and IC₅₀ determination by inhibition ELISA: The binding of H2L7 and five different H2L7 variants to AβpE3-40 monomers AβpE3-42 protofibrils and selectivity versus Aβ1-40 monomers and Aβ1-42 protofibrils was evaluated using inhibition ELISA. H2L7 and its variants demonstrated binding to AβpE3-40 monomer and AβpE3-42 protofibril in solution. None of the antibodies bound Aβ1-40 monomer in solution at concentrations up to 500 nM, suggesting that the corresponding IC₅₀ values were >500 nM. None of the antibodies bound Aβ1-42 protofibrils in solution at concentrations up to 132 nM, suggesting that the corresponding IC₅₀ values were >132 nM. The calculated IC₅₀ values are listed in Table 15.

TABLE 15
Selectivity evaluation using inhibition ELISA (Mean ± SD, n = 2)

| | AβpE3-40 | AβpE3-42 | Aβ1-40 | Aβ1-42 |
|---|---|---|---|---|
| | monomer | protofibril | monomer | protofibril |
| | IC₅₀ | IC₅₀ | IC₅₀ | IC₅₀ |
| Antibody | (nM) | (nM) | (nM) | (nM) |
| H2L7 | 2.35 ± 0.02 | 3.00 ± 0.40 | >500 | >132 |
| H2L7-K12V/R71A | 1.69 ± 0.07 | 3.58 ± 0.58 | >500 | >132 |
| H2L7-R71A | 2.54 ± 0.32 | 4.59 ± 0.19 | >500 | >132 |
| H2L7-K12V/R83T | 2.27 ± 0.47 | 3.71 ± 0.08 | >500 | >132 |
| H2L7-R71A/R83T | 2.77 ± 0.22 | 5.44 ± 0.66 | >500 | >132 |
| H2L7-K12V/R71A/R83T | 2.29 ± 0.21 | 4.90 ± 0.04 | >500 | >132 |

Affinity evaluation and Ko determination by surface plasmon resonance: The binding of H2L7 and the H2L7 variants to AβpE3-40 monomers and AβpE3-42 protofibrils was evaluated by SPR and their K_{D} values were determined.

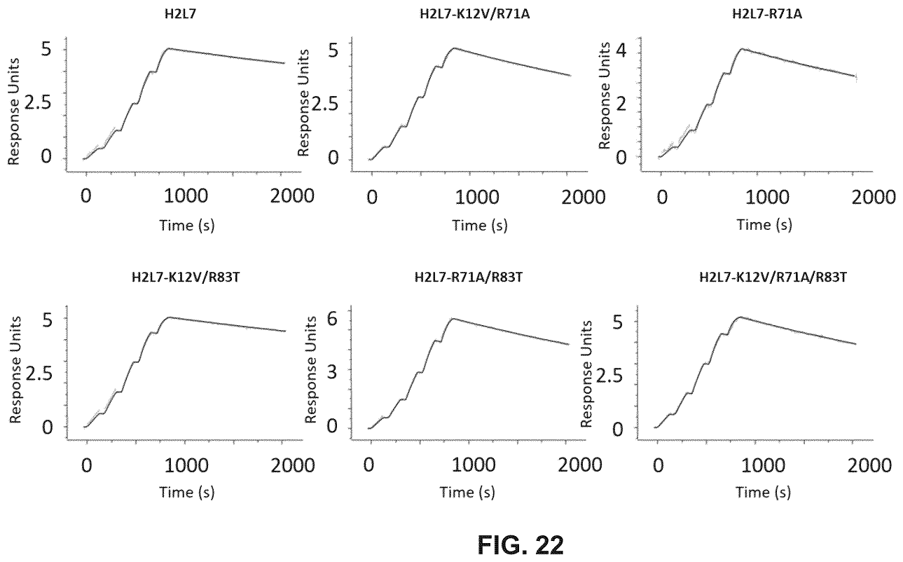

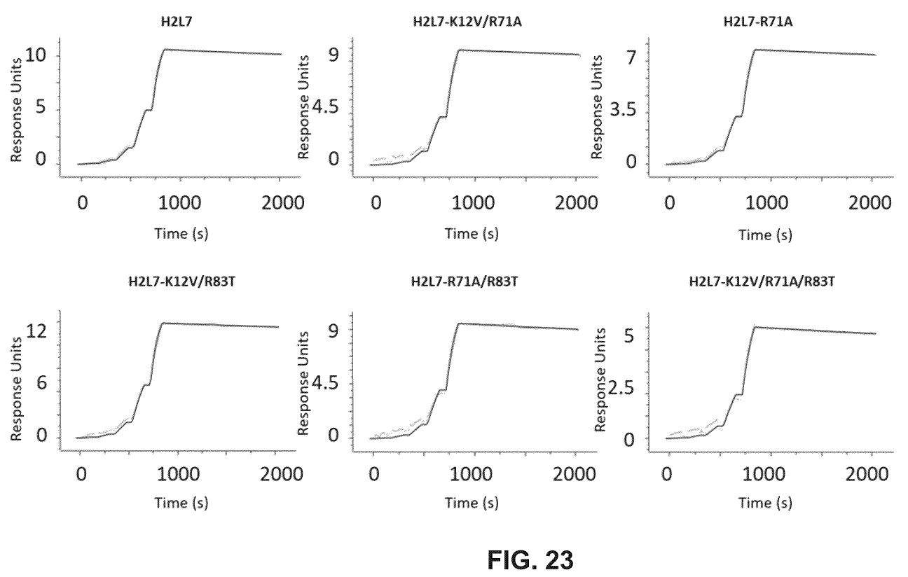

All H2L7 antibody variants demonstrated binding to AβpE3-40 monomers and AβpE3-42 protofibrils. The calculated kₐ, k_{d} and (apparent) K_{D} values are shown in Table 16 and Table 17 below. Representative sensorgrams are shown in FIG. 22 and FIG. 23.

TABLE 16
Summary of SPR analysis of binding to AβpE3-40 monomers

| | AβpE3-40 monomer | | |
|---|---|---|---|
| | kₐ (M⁻¹s⁻¹) | k_{d} (s⁻¹) | K_{D} (nM) |
| Antibody | Mean ± SD | Mean ± SD | Mean ± SD |
| H2L7 | 5.05 ± 0.81 e4 | 1.25 ± 0.24 e−4 | 2.54 ± 0.60 |
| H2L7-K12V/R71A | 5.90 ± 1.09 e4 | 2.33 ± 0.31 e−4 | 4.14 ± 1.23 |
| H2L7-R71A | 5.01 ± 1.04 e4 | 2.25 ± 0.27 e−4 | 4.62 ± 0.88 |
| H2L7-K12V/R83T | 5.62 ± 0.93 e4 | 1.20 ± 0.15 e−4 | 2.20 ± 0.47 |
| H2L7-R71A/R83T | 5.01 ± 1.04 e4 | 2.25 ± 0.27 e−4 | 4.62 ± 0.88 |
| H2L7-K12V/R71A/R83T | 5.87 ± 0.67 e4 | 2.34 ± 0.38 e−4 | 4.04 ± 0.73 |

TABLE 17
Summary of SPR analysis of binding to AβpE3-42 protofibrils

| | AβpE3-42 protofibril | | |
|---|---|---|---|
| | kₐ (M⁻¹s⁻¹) | k_{d} (s⁻¹) | K_{D} (nM) |
| Antibody | Mean ± SD | Mean ± SD | Mean ± SD |
| H2L7 | 1.07 ± 0.08 e5 | 1.79 ± 0.84 e−5 | 173 ± 99.4 |
| H2L7-K12V/R71A | 6.12 ± 0.85 e4 | 2.92 ± 0.68 e−5 | 477 ± 90.6 |
| H2L7-R71A | 6.77 ± 0.70 e4 | 2.54 ± 0.62 e−5 | 376 ± 85.5 |
| H2L7-K12V/R83T | 8.11 ± 0.61 e4 | 1.62 ± 0.48 e−5 | 200 ± 55.7 |
| H2L7-R71A/R83T | 5.74 ± 0.94 e4 | 2.73 ± 0.87 e−5 | 471 ± 95.0 |
| H2L7-K12V/R71A/R83T | 5.76 ± 0.89 e4 | 2.45 ± 1.13 e−5 | 427 ± 190 |

### Example 12

Binding of H2L7 Variant Antibodies to Target in Brain from Human Alzheimer's Disease Patients and Non-Demented Controls

This example describes target binding of H2L7 and of the H2L7 variants generated in Example 10, as tested by immunoprecipitation on human brain extracts and immunohistochemistry on human brain sections from AD patients and NDE controls.

### Materials and Methods

Target binding in human Alzheimer's disease brain extracts by immunoprecipitation: Antibody binding to target in human AD brain was analyzed by immunoprecipitation as described for H2L7 and H9L8 in Example 7.

Target binding in human Alzheimer's disease brain by immunohistochemistry: Immunohistochemistry (IHC) analyses were performed on brain tissue from AD as described for H2L7 and H9L8 in Example 7.

### Results

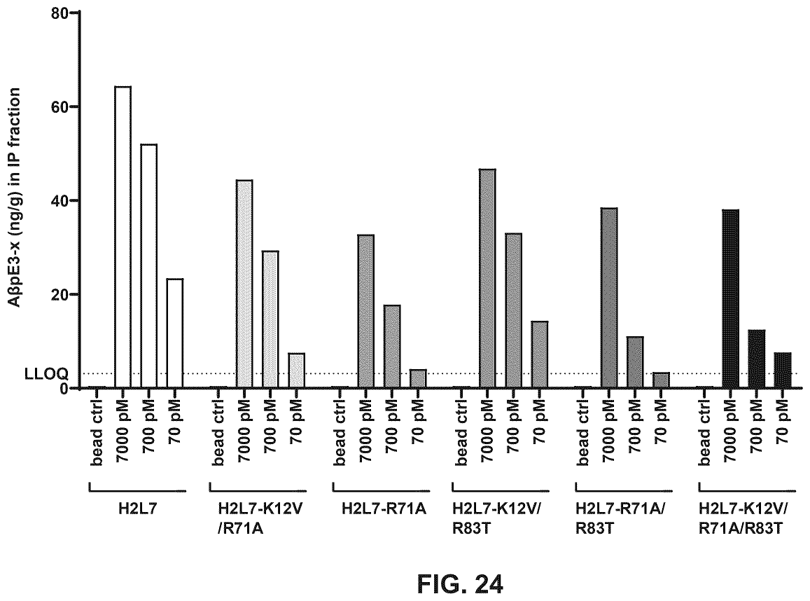

Target binding in human Alzheimer's disease brain extracts by immunoprecipitation: Antibody H2L7 and variants H2L7-K12V/R71A, H2L7-R71A, H2L7-K12V/R83T, H2L7-R71A/R83T and H2L7-K12V/R71A/R83T were tested for their ability to bind to AβpE3 in solution, in human brain extracts from AD patients. Immunoprecipitation (IP) of TBS brain extracts from AD patients demonstrated a concentration dependent IP of AβpE3-x by all tested antibodies (FIG. 24).

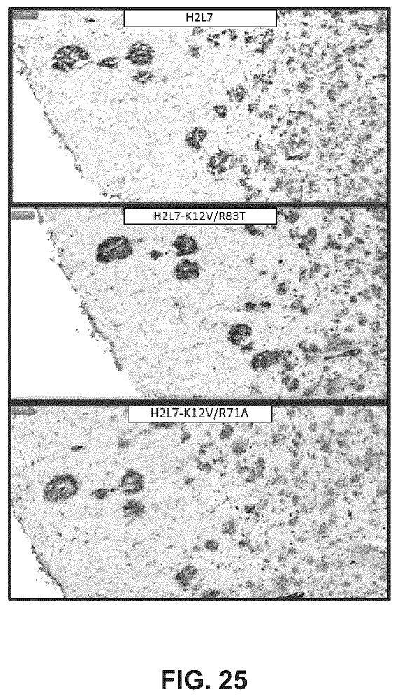

Target binding in human Alzheimer's disease brain by immunohistochemistry: Immunohistochemical staining of brain sections from AD individuals (confirmed to have Aβ pathology by IHC staining with 6E10/4G8, not shown) with H2L7 and variants H2L7-K12V/R71A and H2L7-K12V/R83T resulted in specific binding to core and diffuse plaques in AD brain, with an identical staining pattern. No binding was observed to NDE control brain (data not shown). Representative images from immunostaining with H2L7, H2L7-K12V/R71A, and H2L7-K12V/R83T on adjacent sections from fresh-frozen AD brain are shown in FIG. 25.

### Example 13

### Characterization of Functional Effects of H2L7 Variant Antibodies

This example describes the functional effects of H2L7 variant antibodies generated as described in Example 10. The ability of the variant antibodies to inhibit aggregation of AβpE3 and to clear amyloid plaques in AD brain sections ex vivo was evaluated.

### Materials and Methods

Inhibition of aggregation of Aβ3pE3-42: The effect of the variant antibodies on aggregation of AβpE3-42 monomers was evaluated in an aggregation assay as described for H2L7 and H9L8 in Example 8.

Ex vivo phagocytosis in AD brain: An ex vivo phagocytosis assay was used to investigate whether the variant antibodies can induce plaque clearance by macrophages, as described for H2L7 and H9L8 in Example 8.

### Results

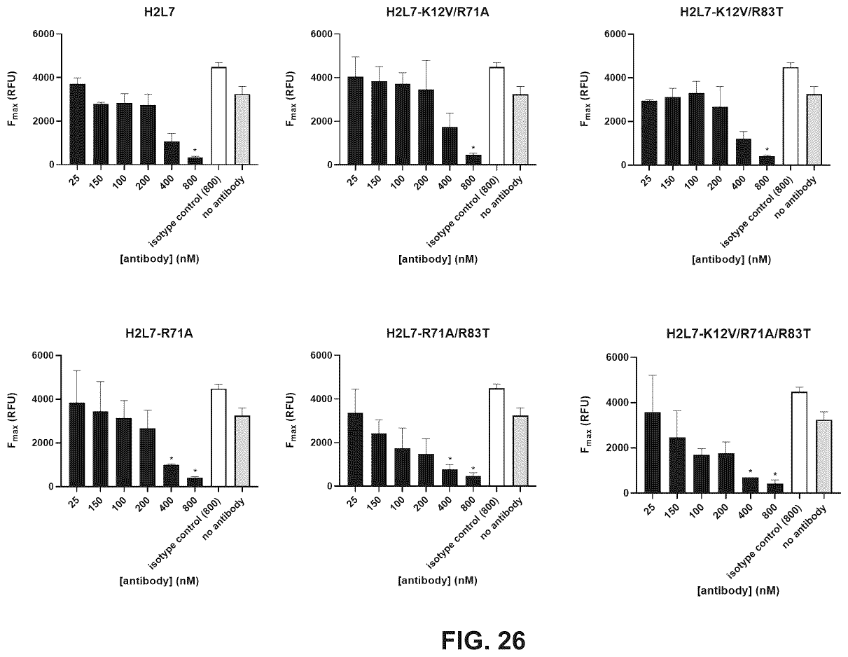

Inhibition of aggregation of Aβ3PE3-42: The ability of the H2L7 variant antibodies to inhibit aggregation of AβpE3-42 was evaluated. All tested antibodies concentration-dependently inhibited fibril formation of AβpE3-42, as demonstrated by the decrease in the maximum ThT fluorescence signal (Fmax) in the presence of the antibodies (FIG. 26).

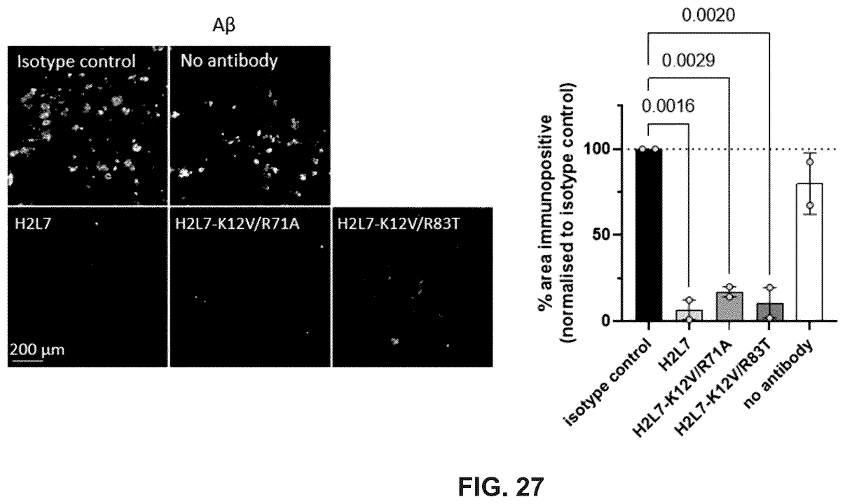

Ex vivo phagocytosis in AD brain: The ability of the H2L7 variant antibodies H2L7-K12V/R71A and H2L7-K12V/R83T to induce clearance of Aβ plaques by macrophages in AD brain was evaluated. Compared to negative control samples pre-incubated without antibodies or with an isotype control IgG1 antibody, Aβ plaques were significantly reduced after pre-incubation with H2L7, H2L7-K12V/R71A and H2L7-K12V/R83T, indicating that the tested antibodies induced plaque clearance by macrophages (FIG. 27).

### Example 14

### Immunogenicity of H2L7 Variant Antibodies

This example describes the assessment of the potential immunogenicity of H2L7 variant antibodies generated as described in Example 10. The ability of the variant antibodies to induce CD4+ T cell responses was evaluated using the Episcreen™ time course assay.

### Materials and Methods

Isolation of peripheral blood mononuclear cells (PBMCs): PBMCs were isolated from healthy community donor buffy coats (from blood drawn within 24 h) obtained under consent from commercial vendors. Cells were separated by density centrifugation using Lymphocyte separation medium (StemCell Technologies Inc, London, UK) and CD8+ T cells were depleted using CD8+RosetteSep™ (StemCell Technologies Inc). Donors were characterized by identifying HLA-DR and HLA-DQ haplotypes to 4-digit resolution by SSO HLA typing (VHBio, Gateshead, UK). T cell responses to the neo-antigen KLH (Invitrogen, Paisley, UK) were also determined. PBMCs were then frozen and stored in the vapour phase of nitrogen until required.

Preparation of samples: Endotoxin levels in the antibody samples were measured using a LAL chromogenic kinetic assay kit (Charles River, Margate, UK) according to the manufacturer's instructions and found to be within the limit acceptable for the assay (<3 EU/mg).

The antibody samples were diluted to 0.6 μM in AIM-V® culture medium (Invitrogen) prior to use (final assay concentration 0.3 μM). KLH was used as a reproducibility control and stored at −20° C. as a 10 mg/ml stock solution in water. For the studies, an aliquot of KLH was thawed immediately before use and diluted to 200 μg/ml in AIM-V® (final concentration 100 μg/ml). A further high immunogenicity control, CEFT (pool of 13 peptides from Pepscan Ltd, Lelystad, The Netherlands) was used as a high responding control and this was stored at −20° C. as a 1.538 mg/ml stock solution and diluted in AIM-V® to 2 μg/ml before use (final concentration 1 μg/ml). Herceptin® (Bionical Ltd, Willington, UK) was used as a negative clinical control and this was stored at −80° C. as a 20 mg/ml stock solution (final assay concentration 50 μg/ml).

Time course proliferation assay: A cohort of 50 donors was selected for the assay. PBMCs from each donor were thawed, counted and viability assessed using an acridine orange (AO) and 4′,6-diamidino-2-phenylindole (DAPI) (Chemometec Ltd, Allerod, Denmark) dye exclusion. Cells were revived in room temperature AIM-V® culture medium, washed and resuspended in AIM-V® to 4-6×10⁶ PBMC/ml for use as the proliferation cell stock. For each donor, bulk cultures were established in which 1 ml of the proliferation cell stock was added to the appropriate wells of a 24 well plate. 1 ml of each sample was added to the PBMCs to give a final sample concentration of 0.3 μM. For each donor, a reproducibility control well (cells incubated with 100 μg/ml KLH), a further high immunogenicity control (cells incubated with 1 μg/ml CEFT peptide pool), a low immunogenicity control (cells incubated with 50 μg/ml Herceptin®) and a culture medium only well were also included. Cultures were incubated for a total of 8 d at 37° C. with 5% CO₂. On days 5, 6, 7 and 8, the cells in each well were gently resuspended by mixing 5× using an electronic pipette and 3×100 μl aliquots transferred to each well of a round bottomed 96 well plate. The cultures were pulsed with 0.75 μCi [3H]-Thymidine (Perkin Elmer, Beaconsfield, UK) in 100 μl AIM-V® culture medium and incubated for a further 18 h before harvesting onto filter mats (Perkin Elmer) using a TomTec Mach III cell harvester. CPM for each well were determined by Meltilex™ (Perkin Elmer) scintillation counting on a 1450 Microbeta Wallac Trilux Liquid Scintillation Counter (Perkin Elmer) in paralux, low background counting.

Assessment of cell viability: On day 7, bulk cultures (previously established for the proliferation assay) were gently resuspended by mixing 5× using an electronic pipette and 50 μl was removed from each well and mixed with 2.5 μl acridine orange (AO) and 4′,6-diamidino-2-phenylindole (DAPI) (Chemometec Ltd) dye exclusion. Cells were then assessed for viability using a NucleoCounter® NC-250™ automated cell analyser (Chemometec Ltd).

Data analysis: For proliferation assays, an empirical threshold of a stimulation index (SI) equal to or greater than 1.9 (SI 1.90) had been previously established, whereby samples inducing responses above this threshold are deemed positive. For proliferation analysis, donors that were positive on at least one time point during the time course assay were deemed positive donors and counted towards the “% Response” parameter.

### Results

The ability of the H2L7 variant antibodies H2L7-K12V/R71A and H2L7-K12V/R83T to induce CD4+ T cell responses was evaluated. Reference antibodies A and B were used for comparison. Reference antibody A comprises the heavy chain amino acid sequence SEQ ID NO:33 and the light chain amino acid sequence SEQ ID NO:34. Reference antibody B comprises the heavy chain amino acid sequence SEQ ID NO:35 and the light chain amino acid sequence SEQ ID NO:36.

EpiScreen™ analysis of the frequency and magnitude of the responses showed that H2L7-K12V/R71A and H2L7-K12V/R83T induced responses slightly above the response for the low immunogenicity control Herceptin® and were therefore considered to have a relatively low risk for immunogenicity. Both reference antibodies A and B induced clearly higher proliferation responses compared to Herceptin® and are therefore considered to have a higher risk of immunogenicity in the clinic.

TABLE 18
Summary of healthy donor proliferation

| Antibody | Mean SI | SD | % Response |
|---|---|---|---|
| H2L7-K12V/R71A | 3.21 | 1.86 | 14 |
| H2L7-K12V/R83T | 3.04 | 1.36 | 18* |
| Reference A | 3.77 | 1.96 | 50 |
| Reference B | 3.97 | 2.55 | 32 |
| Herceptin ® | 3.05 | 0.92 | 10 |
| CEFT | 5.92 | 6.62 | 90 |
| KLH | 11.94 | 12.88 | 100 |

*calculated based on 28 donors

### ITEMIZED LISTING OF EMBODIMENTS

1. An antibody, which has affinity for AβpE3, and in which the six complementarity determining regions of the heavy and light chain variable domain consist of the following amino acid sequences:

VH-CDR1:
(SEQ ID NO: 1)
GX₁TX₂N

wherein
X₁ is selected from Y and F; and
X₂ is selected from L and M;

VH-CDR2:
(SEQ ID NO: 2)
LINPYNGX₃TTYNX₄KFX₅G

wherein
X₃ is selected from I and V
X₄ is selected from P and Q; and
X₅ is selected from M and K;

VH-CDR3:
(SEQ ID NO: 3)
EGNWEGVY
VL-CDR1:
(SEQ ID NO: 4)
X₆SSQSLLDSNGKTYLH

wherein
X₆ is selected from K and R;

VL-CDR2:
(SEQ ID NO: 5)
LVSX-LDS

wherein
X₇ is selected from I and K;

VL-CDR3:
(SEQ ID NO: 6)
VQGTHFPFT,

or an antigen-binding fragment thereof.
2. An antibody or antigen-binding fragment thereof according to item 1, wherein said VH-CDR1, VH-CDR2 and VL-CDR2 regions consist of the following amino acid sequences:

VH-CDR1:
(SEQ ID NO: 7)
GFTMN
VH-CDR2:
(SEQ ID NO: 8)
LINPYNGVTTYNQKFKG
VL-CDR2:
(SEQ ID NO: 9)
LVSILDS.

3. An antibody or antigen-binding fragment thereof according to any preceding item, which comprises a heavy chain variable domain and a light chain variable domain, wherein said heavy chain variable domain comprises an amino acid sequence selected from
i) the group consisting of SEQ ID NO:15-22; and
ii) an amino acid sequence having at least 80% identity to any one of SEQ ID NO:15-22, provided that the three VH-CDR regions consist of SEQ ID NO:7, SEQ ID NO:8 and SEQ ID NO:3.
4. An antibody or antigen-binding fragment thereof according to item 3, wherein the VH amino acid sequence in i) is selected from the group consisting of SEQ ID NO:15-21.
5. An antibody or antigen-binding fragment thereof according to item 4, wherein the VH amino acid sequence in i) is selected from the group consisting of SEQ ID NO:15-16 and 18-21.
6. An antibody or antigen-binding fragment thereof according to item 4, wherein the VH amino acid sequence in i) is selected from the group consisting of SEQ ID NO:15-20.
7. An antibody or antigen-binding fragment thereof according to any one of items 5-6, wherein the VH amino acid sequence in i) is selected from the group consisting of SEQ ID NO:15-16 and 18-20.
8. An antibody or antigen-binding fragment thereof according to item 7, wherein the VH amino acid sequence in i) is selected from the group consisting of SEQ ID NO:15 and SEQ ID NO:18.
9. An antibody or antigen-binding fragment thereof according to item 8, wherein the VH amino acid sequence in i) is SEQ ID NO:18.
10. An antibody or antigen-binding fragment thereof according to any preceding item, in which the amino acid sequence of VL-CDR1 is

(SEQ ID NO: 10)
RSSQSLLDSNGKTYLH.

11. An antibody or antigen-binding fragment thereof according to item 10, which comprises a heavy chain variable domain and a light chain variable domain, wherein said light chain variable domain comprises an amino acid sequence selected from
i) the group consisting of SEQ ID NO:23-24; and
ii) an amino acid sequence having at least 80% identity to any one of SEQ ID NO:23-24, provided that the three VL-CDR regions consist of SEQ ID NO:10, SEQ ID NO:9 and SEQ ID NO:6.
12. An antibody or antigen-binding fragment thereof according to item 11, wherein the VL amino acid sequence in i) is SEQ ID NO:23.
13. An antibody or antigen-binding fragment thereof according to any preceding item, wherein said heavy chain variable domain is as defined in any one of items 3-9 and said light chain variable domain is as defined in any one of items 11-12.
14. An antibody or antigen-binding fragment thereof according to item 13, wherein the heavy chain variable domain and the light chain variable domain are represented by the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
f) a heavy chain variable domain comprising SEQ ID NO:20 and a light chain variable domain comprising SEQ ID NO:23;
g) a heavy chain variable domain comprising SEQ ID NO:21 and a light chain variable domain comprising SEQ ID NO:24;
h) a heavy chain variable domain comprising SEQ ID NO:22 and a light chain variable domain comprising SEQ ID NO:23.
15. An antibody or antigen-binding fragment thereof according to item 14, wherein the heavy chain variable domain and the light chain variable domain are represented by the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
f) a heavy chain variable domain comprising SEQ ID NO:20 and a light chain variable domain comprising SEQ ID NO:23;
g) a heavy chain variable domain comprising SEQ ID NO:21 and a light chain variable domain comprising SEQ ID NO:24.
16. An antibody or antigen-binding fragment thereof according to item 15, wherein the heavy chain variable domain and the light chain variable domain are represented by the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:20 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
g) a heavy chain variable domain comprising SEQ ID NO:21 and a light chain variable domain comprising SEQ ID NO:24.
17. An antibody or antigen-binding fragment thereof according to item 15, wherein the heavy chain variable domain and the light chain variable domain are represented by the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
f) a heavy chain variable domain comprising SEQ ID NO:20 and a light chain variable domain comprising SEQ ID NO:23.
18. An antibody or antigen-binding fragment thereof according to any one of items 16-17, wherein the heavy chain variable domain and the light chain variable domain are represented by the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:20 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23.
19. An antibody or antigen-binding fragment thereof according to item 18, wherein the heavy chain variable domain and the light chain variable domain are represented by the following VH/VL combinations:
a) a heavy chain variable domain comprising SEQ ID NO:18 and a light chain variable domain comprising SEQ ID NO:23;
b) a heavy chain variable domain comprising SEQ ID NO:15 and a light chain variable domain comprising SEQ ID NO:23.
20. An antibody or antigen-binding fragment thereof according to item 19, wherein the heavy chain variable domain and the light chain variable domain are represented by the following VH/VL combination:
a) a heavy chain variable domain comprising SEQ ID NO:18 and a light chain variable domain comprising SEQ ID NO:23.
21. An antibody or antigen-binding fragment thereof according to any one of items 1-9, in which the amino acid sequence of VL-CDR1 is

(SEQ ID NO: 11)
KSSQSLLDSNGKTYLH.

22. An antibody or antigen-binding fragment thereof according to item 21, which comprises a heavy chain variable domain and a light chain variable domain, wherein said heavy chain variable domain comprises an amino acid sequence selected from
i) SEQ ID NO:25; and
ii) an amino acid sequence having at least 80% identity to SEQ ID NO:25, provided that the three VH-CDR regions consist of SEQ ID NO:7, SEQ ID NO:8 and SEQ ID NO:3.
23. An antibody or antigen-binding fragment thereof according to any one of items 21-22, which comprises a heavy chain variable domain and a light chain variable domain, wherein said light chain variable domain comprises an amino acid sequence selected from
i) SEQ ID NO:26; and
ii) an amino acid sequence having at least 80% identity to SEQ ID NO:26, provided that the three VL-CDR regions consist of SEQ ID NO:11, SEQ ID NO:9 and SEQ ID NO:6.
24. An antibody or antigen-binding fragment thereof according to any one of items 22-23, wherein said heavy chain variable domain is as defined in item 22 and said light chain variable domain is as defined in item 23.
25. An antibody or antigen-binding fragment thereof according to item 1, wherein said VH-CDR1, VH-CDR2, VL-CDR1 and VL-CDR2 regions consist of the following amino acid sequences:

VH-CDR1:
(SEQ ID NO: 12)
GYTLN;
VH-CDR2:
(SEQ ID NO: 13)
LINPYNGITTYNPKFMG;
VL-CDR1:
(SEQ ID NO: 11)
KSSQSLLDSNGKTYLH
VL-CDR2:
(SEQ ID NO: 14)
LVSKLDS.

26. An antibody or antigen-binding fragment thereof according to item 25, which comprises a heavy chain variable domain and a light chain variable domain, wherein said heavy chain variable domain comprises an amino acid sequence selected from
i) SEQ ID NO:27; and
ii) an amino acid sequence having at least 80% identity to SEQ ID NO:27, provided that the three VH-CDR regions consist of SEQ ID NO:12, SEQ ID NO:13 and SEQ ID NO:3.
27. An antibody or antigen-binding fragment thereof according to any one of items 25-26, which comprises a heavy chain variable domain and a light chain variable domain, wherein said light chain variable domain comprises an amino acid sequence selected from
i) SEQ ID NO:28; and
ii) an amino acid sequence having at least 80% identity to SEQ ID NO:28, provided that the three VL-CDR regions consist of SEQ ID NO:11, SEQ ID NO:14 and SEQ ID NO:6.
28. An antibody or antigen-binding fragment thereof according to any one of items 26-27, wherein said heavy chain variable domain is as defined in item 26 and said light chain variable domain is as defined in item 27.
29. An antibody or antigen-binding fragment thereof according to any preceding item, wherein said AβpE3 is in a form selected from the group consisting of monomers, protofibrils, fibrils and plaques.
30. An antibody or antigen-binding fragment thereof according to any preceding item, which has a higher binding affinity for AβpE3 monomers than for Aβ1-X monomers.
31. An antibody or antigen-binding fragment thereof according to item 30, which has at least 2× higher binding affinity for AβpE3 monomers than for Aβ1-X monomers, such as at least 10× higher, such as at least 100× higher, such as at least 1000× higher, such as at least 3000× higher binding affinity.
32. An antibody or antigen-binding fragment thereof according to any preceding item, which has a higher binding affinity for protofibrils comprising AβpE3 than for AβpE3 monomers.
33. An antibody or antigen-binding fragment thereof according to item 24, which has at least 2× higher binding affinity for protofibrils comprising AβpE3 than for AβpE3 monomers, such as at least 10× higher, such as at least 40× higher, such as at least 100× higher, such as at least 200× higher binding affinity.
34. An antibody or antigen-binding fragment thereof according to any preceding item, which has a binding affinity for protofibrils comprising AβpE3 that corresponds to a K_{D} value of no more than 1 nM, such as between 1 and 200 pM, such as between 10 and 100 pM, as determined by surface plasmon resonance.
35. An antibody or antigen-binding fragment thereof according to any preceding item, which has a binding affinity for AβpE3 monomers that corresponds to a K_{D} value of no more than 100 nM, such as between 0.1 and 50 nM, such as between 0.5 and 10 nM, as determined by surface plasmon resonance.
36. An antigen-binding fragment according to any preceding item, which is selected from the group consisting of Fab fragments, Fab′ fragments, F(ab′)₂ fragments, Fv fragments, single chain Fv fragments, (scFv)₂ and domain antibodies.
37. An antibody or antigen-binding fragment thereof according to any preceding item, which is of IgG class.
38. An antibody or antigen-binding fragment thereof according to item 37, wherein said IgG class is selected from the group consisting of IgG1 and IgG4.
39. An antibody or antigen-binding fragment thereof according to any preceding item, which is monoclonal.
40. An antibody or antigen-binding fragment thereof according to any preceding item, which is selected from the group consisting of human antibodies and fragments thereof; humanized antibodies and fragments thereof; and antibodies and fragments thereof that have been mutated to reduce the antigenicity thereof in humans.
41. Pharmaceutical composition, comprising an antibody or antigen-binding fragment thereof according to any preceding item and a pharmaceutically acceptable carrier or excipient.
42. An antibody or antigen-binding fragment thereof according to any one of items 1-40 or a composition according to item 41 for use in treatment, such as for use in therapeutic treatment or for use in prophylactic treatment.
43. An antibody or antigen-binding fragment thereof according to any one of items 1-40 or composition according to item 41 for use in diagnosis in vivo or prognosis in vivo.
44. An antibody or antigen-binding fragment thereof or composition for use according to any one of items 42-43, wherein said therapy, prophylaxis, in vivo diagnosis or in vivo prognosis is with respect to a neurodegenerative disorder associated with amyloid beta peptide aggregation, for example a disorder selected from the group consisting of Alzheimer's disease (AD) (including familial AD and sporadic AD), mild cognitive impairment (MCI), Lewy body dementia, neurodegeneration in Down's syndrome, cerebral amyloid angiopathy (CAA), hereditary cerebral hemorrhage with amyloidosis (Dutch type), progressive supranuclear palsy, multiple sclerosis, Creutzfeld-Jacob disease, cerebral amyloid angiopathy, Parkinson's disease, amyotrophic lateral sclerosis, cataract due to Aβ deposition, traumatic brain injury with an accumulation of Aβ, adult onset diabetes, senile cardiac amyloidosis and macular degeneration.
45. An antibody or antigen-binding fragment thereof for use according to item 44, wherein said neurodegenerative disorder is Alzheimer's disease.
46. A method of therapeutic or prophylactic treatment of a mammal having, or being at risk of developing, a neurodegenerative disorder, said method comprising administering to said mammal a therapeutically effective amount of an antibody or antigen-binding fragment thereof according to any one of items 1-40 or a composition according to item 41.
47. A method according to item 46, wherein said neurodegenerative disorder is a disorder associated with amyloid beta peptide aggregation, for example a disorder selected from the group consisting of Alzheimer's disease (AD) (including familial AD and sporadic AD), mild cognitive impairment (MCI), Lewy body dementia, neurodegeneration in Down's syndrome, cerebral amyloid angiopathy (CAA), hereditary cerebral hemorrhage with amyloidosis (Dutch type), progressive supranuclear palsy, multiple sclerosis, Creutzfeld-Jacob disease, cerebral amyloid angiopathy, Parkinson's disease, amyotrophic lateral sclerosis, cataract due to Aβ deposition, traumatic brain injury with an accumulation of Aβ, adult onset diabetes, senile cardiac amyloidosis and macular degeneration.
48. A method according to item 47, wherein said neurodegenerative disorder is Alzheimer's disease.
49. A method of detecting AβpE3 peptides in vitro, comprising providing a sample suspected to contain Aβ peptides, contacting said sample with an antibody or antigen-binding fragment thereof according to any one of items 1-40, and detecting the binding of said protein to indicate the presence of AβpE3 peptides in the sample.
50. A method of determining the amount of AβpE3 peptides present in a subject, comprising the steps of:
a) contacting the subject, or a sample isolated from the subject, with an antibody or antigen-binding fragment thereof according to any one of items 1-40 or a composition according to item 41, and
b) obtaining a value corresponding to the amount of the antibody or antigen-binding fragment thereof or composition that has bound in said subject or to said sample.
51. Method according to item 50, further comprising a step of comparing said value to a reference.

1. An antibody, which has affinity for AβpE3, and in which the six complementarity determining regions of the heavy and light chain variable domain consist of the following amino acid sequences:
VH-CDR1:
(SEQ ID NO: 1)
GX₁TX₂N
wherein
X₁ is selected from Y and F; and
X₂ is selected from L and M;
VH-CDR2:
(SEQ ID NO: 2)
LINPYNGX₃TTYNX₄KFX₅G
wherein
X₃ is selected from I and V
X₄ is selected from P and Q; and
X₅ is selected from M and K;
VH-CDR3:
(SEQ ID NO: 3)
EGNWEGVY
VL-CDR1:
(SEQ ID NO: 4)
X₆SSQSLLDSNGKTYLH
wherein
X₆ is selected from K and R;
VL-CDR2:
(SEQ ID NO: 5)
LVSX-LDS
wherein
X₇ is selected from I and K;
VL-CDR3:
(SEQ ID NO: 6)
VQGTHFPFT,
or an antigen-binding fragment thereof.

2. An antibody or antigen-binding fragment thereof according to claim 1, wherein said VH-CDR1, VH-CDR2 and VL-CDR2 regions consist of the following amino acid sequences:
VH-CDR1:
(SEQ ID NO: 7)
GFTMN
VH-CDR2:
(SEQ ID NO: 8)
LINPYNGVTTYNQKFKG
VL-CDR2:
(SEQ ID NO: 9)
LVSILDS.

3. An antibody or antigen-binding fragment thereof according to any preceding claim, which comprises a heavy chain variable domain and a light chain variable domain, wherein said heavy chain variable domain comprises an amino acid sequence selected from
i) the group consisting of SEQ ID NO:15-22; and
ii) an amino acid sequence having at least 80% identity to any one of SEQ ID NO:15-22, provided that the three VH-CDR regions consist of SEQ ID NO:7, SEQ ID NO:8 and SEQ ID NO:3.

4. An antibody or antigen-binding fragment thereof according to any preceding claim, in which the amino acid sequence of VL-CDR1 is
(SEQ ID NO: 10)
RSSQSLLDSNGKTYLH.

5. An antibody or antigen-binding fragment thereof according to claim 4, which comprises a heavy chain variable domain and a light chain variable domain, wherein said light chain variable domain comprises an amino acid sequence selected from
i) the group consisting of SEQ ID NO:23-24; and
ii) an amino acid sequence having at least 80% identity to any one of SEQ ID NO:23-24, provided that the three VL-CDR regions consist of SEQ ID NO:10, SEQ ID NO:9 and SEQ ID NO:6.

6. An antibody or antigen-binding fragment thereof according to any preceding claim, wherein the heavy chain variable domain and the light chain variable domain are represented by the following VH/VL combinations:
a) a heavy chain variable domain comprising SEQ ID NO:15 and a light chain variable domain comprising SEQ ID NO:23;
b) a heavy chain variable domain comprising SEQ ID NO:16 and a light chain variable domain comprising SEQ ID NO:23;
c) a heavy chain variable domain comprising SEQ ID NO:17 and a light chain variable domain comprising SEQ ID NO:23;
d) a heavy chain variable domain comprising SEQ ID NO:18 and a light chain variable domain comprising SEQ ID NO:23;
e) a heavy chain variable domain comprising SEQ ID NO:19 and a light chain variable domain comprising SEQ ID NO:23;
f) a heavy chain variable domain comprising SEQ ID NO:20 and a light chain variable domain comprising SEQ ID NO:23;
g) a heavy chain variable domain comprising SEQ ID NO:21 and a light chain variable domain comprising SEQ ID NO:24;
h) a heavy chain variable domain comprising SEQ ID NO:22 and a light chain variable domain comprising SEQ ID NO:23.

7. An antibody or antigen-binding fragment thereof according to any preceding claim, wherein said AβpE3 is in a form selected from the group consisting of monomers, protofibrils, fibrils and plaques.

8. An antibody or antigen-binding fragment thereof according to any preceding claim, which has a binding affinity for protofibrils comprising AβpE3 that corresponds to a K_{D} value of no more than 1 nM, such as between 1 and 200 μM, such as between 10 and 100 μM, as determined by surface plasmon resonance.

9. An antibody or antigen-binding fragment thereof according to any preceding claim, which has a binding affinity for AβpE3 monomers that corresponds to a K_{D} value of no more than 100 nM, such as between 0.1 and 50 nM, such as between 0.5 and 10 nM, as determined by surface plasmon resonance.

10. Pharmaceutical composition, comprising an antibody or antigen-binding fragment thereof according to any preceding claim and a pharmaceutically acceptable carrier or excipient.

11. An antibody or antigen-binding fragment thereof according to any one of claims 1-9 or a composition according to claim 10 for use in treatment, such as for use in therapeutic treatment or for use in prophylactic treatment.

12. An antibody or antigen-binding fragment thereof according to any one of claims 1-9 or composition according to claim 10 for use in diagnosis in vivo or prognosis in vivo.

13. An antibody or antigen-binding fragment thereof or composition for use according to any one of claims 11-12, wherein said therapy, prophylaxis, in vivo diagnosis or in vivo prognosis is with respect to a neurodegenerative disorder associated with amyloid beta peptide aggregation, for example a disorder selected from the group consisting of Alzheimer's disease (AD) (including familial AD and sporadic AD), mild cognitive impairment (MCI), Lewy body dementia, neurodegeneration in Down's syndrome, cerebral amyloid angiopathy (CAA), hereditary cerebral hemorrhage with amyloidosis (Dutch type), progressive supranuclear palsy, multiple sclerosis, Creutzfeld-Jacob disease, cerebral amyloid angiopathy, Parkinson's disease, amyotrophic lateral sclerosis, cataract due to Aβ deposition, traumatic brain injury with an accumulation of Aβ, adult onset diabetes, senile cardiac amyloidosis and macular degeneration.

14. An antibody or antigen-binding fragment thereof for use according to claim 13, wherein said neurodegenerative disorder is Alzheimer's disease.
